(19) United States
(12) Patent Application Publication
(10) Pub. No.: US 2022/0305013 A1
(43) Pub. Date: Sep. 29, 2022

(54) STABLE COLLOIDAL DRUG AGGREGATES AND METHODS OF MANUFACTURE AND USE THEREOF

(71) Applicants: THE GOVERNING COUNCIL OF THE UNIVERSITY OF TORONTO, Toronto (CA); THE REGENTS OF THE UNIVERSITY OF CALIFORNIA, Oakland (US)

(72) Inventors: Molly S. SHOICHET, Toronto (CA); Ahil N. GANESH, Richmond Hill (CA); Eric N. DONDERS, Deep River (CA); Brian K. SHOICHET, San Francisco, CA (US); Hayarpi TOROSYAN, San Francisco, CA (US)

(21) Appl. No.: 17/619,837

(22) PCT Filed: Jun. 18, 2020
(86) PCT No.: PCT/CA2020/050845

§ 371 Date: Dec. 16, 2021

(30) Foreign Application Priority Data
Jun. 18, 2019 (GB) 1908716.2

(51) Int. Cl. A61K 31/506; A61K 9/10; A61K 47/42; A61K 31/4174; A61K 31/438; A61K 47/28; A61K 47/34; A61K 31/566; A61K 31/44

(52) U.S. Cl. CPC A61K 31/506; A61K 9/10; A61K 47/42; A61K 31/4174; A61K 31/438; A61K 47/28; A61K 47/34; A61K 31/566; A61K 31/44

(57) ABSTRACT

The present application provides a colloid drug aggregate composition and methods of use and manufacture thereof. While the formation of colloidal aggregates leads to artifacts in early drug discovery, their composition makes them attractive as nanoparticle formulations for targeted drug delivery. The present application provides an acid-responsive composition comprising: a colloidal aggregate of one or more drugs and a stabilizing agent, wherein the colloidal aggregate disrupts, dissolves or disassembles when the acid-responsive composition is in an acid environment having a pH of less than 7.4. The colloidal aggregate of the composition will disassemble upon contact with acid or upon introduction to an acidic environment, such as is found in the endosomes of cells. This approach makes this composition an attractive vehicle for drug delivery to a target site in a subject or to cells.

### STATEMENT REGARDING FEDERALLY SPONSORED RESEARCH

This invention was made with government support under grant no. GM071630 awarded by The National Institutes of Health. The government has certain rights in the invention.

### CROSS-REFERENCE TO RELATED APPLICATIONS

The present application claims priority from United Kingdom Patent Application No. GB1908716.2, filed Jun. 18, 2019, which is incorporated herein in its entirety.

### FIELD OF THE INVENTION

The present application pertains to the field of pharmaceuticals. More particularly, the present application relates to colloidal drug aggregates, and methods of manufacture and uses thereof.

### INTRODUCTION

Some small-molecule drugs spontaneously self-assemble under aqueous conditions to form colloidal aggregates.¹⁻³ The formation of these colloids is governed by a critical aggregation concentration (CAC) and generally occurs when water is added to a solution of drug in a water-miscible organic solvent.⁴ Although colloidal drug aggregates are best known for causing false hits in early drug discovery,⁵⁻⁷ recent efforts have aimed to stabilize these drug-rich particles for delivery.⁸⁻¹⁰

Development of stable colloidal drug aggregates has paradoxically created a new problem: colloid-associated drug does not permeate the cell membrane to interact with intracellular targets.¹¹⁻¹² One method to overcome this issue is to disrupt the particles to yield drug monomers.^{12, 13} Colloid dissolution has traditionally been accomplished by adding detergent; however, this strategy is more useful in vitro than in vivo, where toxic excipients are dose-limited.

A need remains for colloidal drug formulations that do not require chemical modification for aggregation and that minimize or avoid the use of toxic excipients for stabilization.

The above information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present application is to provide stable colloidal drug aggregates and methods of manufacture and use thereof. In accordance with an aspect of the present application, there is provided an acid-responsive composition comprising: a colloidal aggregate of one or more drugs and a stabilizing agent, wherein the colloidal aggregate disrupts, dissolves or disassembles when the acid-responsive composition is in an acid environment having a pH of less than 7.4 (such as, a pH of less than about 6.5, or less than about 6, or less than about 5.5, or less than about 5, or less than about 4.5, or less than about 4). In accordance with one embodiment, the acid-responsive composition comprises: a colloidal aggregate of one or more drugs and a stabilizing agent, wherein (a) at least one of the one or more drugs is an ionizable drug or ionizable drug analogue, wherein the conjugate acid of the ionizable drug or drug analogue has a pKa of at least 4, preferably at least 4.5 or at least 5, or at least 5.5, or at least 6, or at least 6.5; (b) the stabilizing agent is an acid-responsive stabilizing agent such that the stabilizing agent undergoes a morphological and/or functional change when pH is reduced to less than about 6.5, or less than about 6, or less than about 5.5, or less than about 5, or less than about 4.5, or less than about 4; or (c) both (a) and (b).

In accordance with an embodiment of the present application, there is provided an acid-responsive composition comprising: a colloidal aggregate of one or more ionizable drugs or ionizable drug analogues; and a stabilizing agent, wherein the conjugate acid of the ionizable drug or drug analogue has a pKa of at least 4, preferably at least 4.5 or at least 5, or at least 5.5, or at least 6, or at least 6.5. In another embodiment of the present application, there is provided an acid-responsive composition comprising a colloidal aggregator, which is either an ionizable or non-ionizable drug or drug analogue; and an acid-responsive stabilizing agent that undergoes a morphological and/or functional change when the pH is reduced to less than about 6.5, or less than about 6, or less than about 5.5, or less than about 5, or less than about 4.5, or less than about 4.

In certain embodiments the stabilizing agent is a protein (e.g. an antibody or antibody fragment), a polymer, a colloid-forming compound (e.g., vitamin E) or another colloid-forming drug (e.g., fulvestrant). In the example in which the stabilizing agent is another colloid-forming drug, the other colloid-forming drug is, optionally, not ionizable at a pH of 4 or less. Optionally, the stabilizing protein is IgG, trastuzumab, albumin, transferrin, or an attenuated bacterial toxin (such as attenuated diphtheria toxin or derivatives thereof). Suitable stabilizing polymers include polymeric surfactants, such as, but not limited to, UP80, PLAC-PEG, Brij 58, F127, Vitamin E-PEG, F68, or Brij L23.

In accordance with certain embodiments, the stabilizing agent compromises the integrity of plasma membranes in acidic environments (e.g., the stomach or a lysosome or an endosome of a cell), thereby allowing colloidal aggregates or their contents to be transported out of the acidic environment.

In accordance with certain embodiments, the colloidal aggregate is disrupted upon contact of the composition with an acid or introduction of the composition to an acidic environment (e.g., the stomach or a lysosome or an endosome of a cell), and the one or more drug or drug analogue is released as a result of the disruption of the colloid.

In accordance with certain embodiments, the colloidal aggregate comprises a targeting compound for delivery of the composition to a target site. The targeting compound can be, for example, a protein (e.g., transferrin), an antibody (e.g. trastuzumab), an attenuated bacterial toxin (such as attenuated diphtheria toxin or derivatives thereof), or another molecule that selectively binds to a cell receptor. Optionally, the targeting compound also functions as the stabilizing agent, or acts together with another stabilizing agent to aid in stabilization of the colloidal aggregate.

In a specific embodiment, the ionizable drug is lapatinib and the composition further comprises a stabilizing compound, which is fulvestrant, and a targeting compound, which is transferrin.

In another specific embodiment, the ionizable drug is lapatinib and the composition further comprises a stabilizing compound, which is a combination of fulvestrant and a polymeric surfactant (e.g., PLAC-PEG).

In accordance with certain embodiments, the colloidal drug aggregate composition comprises an ionizable drug analogue that comprises a drug molecule (e.g., sorafenib or fulvestrant) chemically modified to include an ionizable moiety. Optionally, the ionizable drug analogue is itself pharmaceutically active. Alternatively, the analogue functions as a prodrug such that following ionization and release from the colloid, the analogue is modified (e.g., by cleavage of the ionized moiety) in vivo to form the active drug molecule.

Another aspect of the present application provides a method of drug therapy comprising administration of the colloidal drug aggregate composition to a subject in need thereof.

### BRIEF DESCRIPTION OF FIGURES AND TABLES

For a better understanding of the application as described herein, as well as other aspects and further features thereof, reference is made to the following description which is to be used in conjunction with the accompanying drawings and tables.

FIG. 1: Curve-fitting algorithm identifies the critical aggregation concentration of lapatinib at A) pH 7.4 and B) pH 5.5. Lapatinib fluorescence is proportional to the amount of colloidal lapatinib, which increases linearly with concentration once the CAC is reached.

FIG. 2: Lapatinib is not stabilized by polymeric excipients. Colloidal lapatinib at 50 μM was formulated with 0.01 mg/mL stabilizer in water and incubated at 37° C. DLS characterization of A) size and B) scattering intensity shows precipitation over 24 h, even in formulations that were initially stable.

FIG. 3: Lapatinib is not stabilized in PBS by co-aggregation with Congo red (CR). Colloidal lapatinib at 50 μM was formulated with varying amounts of Congo red in PBS and incubated at 37° C. DLS characterization of A) size and B) scattering intensity shows precipitation over 24 h, even in formulations that were initially stable (n=3, mean±SD).

FIG. 4: Transmission electron micrographs of colloidal drug aggregates. Colloids were formulated with 25 μM each of fulvestrant and lapatinib with 0.01 mg/mL UP80 in PBS. Three representative darkfield images show the spherical morphology and 100-200 nm size of these colloidal drug aggregates.

FIG. 5: Zeta potential of stabilized lapatinib and fulvestrant colloids (fulvestrant alone shown in the lower bars, with negative zeta potential). Bare lapatinib-containing colloids have a highly positive surface charge, which is mitigated by stabilizers (n=3, mean±SD).

FIG. 6: Colloidal lapatinib is stabilized by co-aggregation with fulvestrant and polymeric surfactant. Colloidal drug aggregates were formulated with 50 μM total drug, and 0.01 mg mL⁻¹ poly(D,L-lactide-co-2-methyl-2-carboxytrimethylene carbonate)-graft-poly(ethylene glycol) (PLAC-PEG) in phosphate buffered saline. A 1:3 mol ratio of lapatinib:fulvestrant was selected for its stability against A) growth and B) precipitation as measured by dynamic light scattering. C) Fluorescence measurement (λₑₓ=340 nm, λₑₘ=450 nm) confirmed the presence of lapatinib in these co-colloids (n=3, mean±SD).

FIG. 7: Colloidal stability characterized by dynamic light scattering, comparing PLAC-PEG-stabilized co-colloids containing different ratios of fulvestrant and lapatinib. Colloids were formulated in PBS with 50 μM total drug and 0.01 mg/mL PLAC-PEG. The stable lapatinib-fulvestrant colloids were confirmed to be relatively monodisperse (PDI<0.2) by DLS (n=3, mean±SD).

FIG. 8: Stability characterization by dynamic light scattering of lapatinib and fulvestrant co-colloids stabilized with different amounts of transferrin. Concentrations of 12.5 μM lapatinib and 37.5 μM fulvestrant were used in this study, and transferrin was introduced in the water addition step. DLS analysis of A) hydrodynamic diameter, B) scattering intensity, and C) size dispersity shows that transferrin stabilizes these colloids in a concentration-dependent manner (n=3, mean±SD).

FIG. 9: Stability characterization by dynamic light scattering (DLS) of lapatinib and fulvestrant co-colloids stabilized with transferrin. Co-colloids comprised concentrations of 50 μM (29 μg/mL) lapatinib and 150 μM (91 μg/mL) fulvestrant, and 100 μg/mL transferrin. DLS analyses show that transferrin stabilizes these high-concentration colloids: A) hydrodynamic diameter, B) scattering intensity, and C) polydispersity index (PDI) (n=3, mean±SD).

FIG. 10: Stability characterization by dynamic light scattering of colloids formulated in cell culture media. Colloids were formulated as specified in Table 4. DLS analysis of A) hydrodynamic diameter, B) scattering intensity, and C) polydispersity index (PDI) demonstrate stability in this environment (n=3, mean±SD).

FIG. 11: Colloids persist after acidification. Colloids were formulated with 37.5 μM fulvestrant, 12.5 μM lapatinib, and either 0.05 mg/mL transferrin or 0.01 mg/mL PLAC-PEG, as indicated in the legend. Dynamic light scattering analyses demonstrate persistence of the colloids after acidification: A) hydrodynamic diameter, B) scattering intensity, and C) polydispersity index (PDI) (n=3, mean±SD).

FIG. 12: Lapatinib release from stable lapatinib-fulvestrant co-colloids is triggered by acidic conditions. Colloids were formulated with 12.5 μM lapatinib, 37.5 μM fulvestrant, and 0.01 mg mL⁻¹ poly(D,L-lactide-co-2-methyl-2-carboxytrimethylene carbonate)-graft-poly(ethylene glycol) (PLAC-PEG) in PBS. A) Lapatinib was released from the co-colloids with decreasing pH whereas B) fulvestrant was retained within the colloids. C) Lapatinib release from co-colloids was triggered by acidification to pH 5.5 (colored arrows) whereas D) fulvestrant release was unaffected (n≥3, mean±SD, one-way ANOVA with Tukey's post-hoc test, ns p>0.05, ****p<0.0001 compared to initial release at pH 7.4).

FIG. 13: Lapatinib release from stable lapatinib-fulvestrant co-colloids is triggered by acidic conditions. Colloids were formulated with 50 μM lapatinib, 150 μM fulvestrant, and 0.04 mg/mL PLAC-PEG in PBS. A) Lapatinib was released from the co-colloids with decreasing pH whereas B) fulvestrant was retained within the colloids.

FIG. 14: Lapatinib release from co-colloids is triggered by acidic conditions. Colloidal drug aggregates were formulated with 12.5 μM lapatinib, 37.5 μM fulvestrant, and 0.01 mg/mL PLAC-PEG in PBS supplemented with 10% FBS. The pH of these formulations were adjusted, followed by centrifugation to pellet out the colloids. Finally, the concentration of drug remaining in the supernatant was quantified. A) Lapatinib dissolved from the co-colloids under acidic conditions, whereas B) fulvestrant did not (n≥3, one-way ANOVA with Tukey's post-hoc test, ns p>0.05, *p<0.05, ***p<0.001 compared to pH 7.4, mean±SD). These colloids were also incubated over time and acidified to pH 5.5 as indicated by the arrows; under these conditions C) lapatinib release was rapid and persisted, whereas D) the concentration of free fulvestrant was unaffected (n=3, one-way ANOVAs with Tukey's post-hoc test, *p<0.05, **p<0.01, ****p<0.0001 compared to initial time point, mean±SD).

FIG. 15: Decoration of colloidal drug aggregates with transferrin enhances their endocytosis. MDA-MB-231-H2N cells were treated with colloids (Table 4) with the addition of 500 nM hydrophobic BODIPY dye. Images: cells were treated with lapatinib and fulvestrant colloids stabilized with either A) transferrin or B) poly(D,L-lactide-co-2-methyl-2-carboxytrimethylene carbonate)-graft-poly(ethylene glycol) (PLAC-PEG) for 1 h followed by fresh media for 23 h. The cells were labeled with CellMask™ Green (1×, 5 min) and imaged under live cell conditions. Transferrin-stabilized colloids were visualized as punctate structures inside the cells whereas PLAC-PEG-stabilized colloids were not observed inside the cells. C) Cells were treated for 3 h, and fluorescence of the BODIPY dye inside the cells was then analyzed by flow cytometry (n=3 biological replicates, mean±SD, two-way ANOVA with Tukey's post-hoc test, ***p<0.001, ****p<0.0001 compared to all other groups).

FIG. 16: Colloids are visualized in punctate structures in cells only when stabilized by transferrin. Treatments were formulated as specified in Table 4 with the addition of 500 nM BODIPY dye (all except “Blank”). MDA-MB-231-H2N cells were treated in serum-free media for 3 h, then washed, fixed, and imaged. These representative confocal images show 450 nm lapatinib fluorescence (blue), 575 nm colloid dye fluorescence (green), and 488 nm transmission showing cell outlines.

FIG. 17: Lapatinib-fulvestrant colloids are cytotoxic after endocytosis and subsequent endosome disruption. Colloidal drug aggregates were formulated as described in Table 4, with the addition of 2 μM 7-aminoactinomycin D (7-AAD) to monitor endosome disruption in those experiments. Colloids composed of 50 μM lapatinib and 150 μM fulvestrant and stabilized with transferrin resulted in greater A) cytotoxicity and B) endosomal disruption (measured by amount of nuclear dye) than either co-colloids stabilized with PLAC-PEG or colloids of fulvestrant alone (150 μM fulvestrant plus stabilizer) (n=9 biological replicates and separate colloid formulations for the toxicity experiment and n=3 for the endosome disruption experiment, mean±SD, two-way ANOVA with Tukey's post-hoc test, *p<0.05, **p<0.01, ****p<0.0001). C) The top panel of images shows staining for Lapatinib and the bottom panel of images shows staining for 7-AAD. Nuclear 7-AAD fluorescence is visible after treatment with lapatinib-fulvestrant colloids stabilized with transferrin, demonstrating its endosomal escape. Permeabilized cells accumulate 7-AAD in their nuclei as expected whereas healthy cells treated with 7-AAD alone do not. The presence of cells in each region of interest was verified using the transmission channel prior to capturing these images.

FIG. 18: Control images visualize 7-AAD nuclear fluorescence (red) in colloid-treated MDA-MB-231-H2N cells. Cells were treated with conditions outlined in Table 4 supplemented with 2 μM 7-AAD. The presence of cells in each region of interest was verified using the transmission channel prior to capturing these images.

FIG. 19: MDA-MB-231-H2N cells are still viable immediately after 3 h of treatment with colloids. Cells were treated identically to those used in viability and flow cytometry experiments depicted in FIG. 17, then immediately assessed for viability (n=3, one-way ANOVA with Tukey's post-hoc test, ns p>0.05, mean±SD).

FIG. 20: Lapatinib diffusion across an artificial lipid membrane is decreased under acidic conditions. Colloidal drug aggregates were formulated with 50 μM lapatinib, 150 μM fulvestrant, and 0.04 mg/mL PLAC-PEG in media containing 10% FBS. After formulation, the pH was adjusted, and the colloid solution was pipetted into the donor compartment of the plate. A) Lapatinib and B) fulvestrant concentrations in the receiver compartment after 6 h of incubation at 37° C. are shown (n=5, one-way ANOVA with Tukey's post-hoc test, ns p>0.05, **p<0.01, ***p<0.001, ****p<0.0001, mean±SD).

FIG. 21: Most lapatinib that is released at low pH is ionized. Acid-base theory was used to calculate the amount of released lapatinib shown in FIG. 12C that is uncharged (n≥9, one-way ANOVA with Tukey's post-hoc test, ***p<0.001, mean±SD).

FIG. 22: Characterization of transferrin displacement by serum proteins adsorbing to colloidal drug aggregates. Colloids were formulated with 37.5 μM fulvestrant, 12.5 μM lapatinib, and 0.01 mg/mL Alexa Fluor™ 488 labelled transferrin. Measurement of free transferrin revealed that serum proteins displace transferrin from the surface of the colloids (n=3, mean±SD).

FIG. 23: Trastuzumab-stabilized clotrimazole colloids were disrupted by acid. (A) Colloids of fulvestrant (non-responsive) or clotrimazole (acid-responsive) were formulated at 50 μM and stabilized with 3 μM trastuzumab at pH 7 followed by acidification of pH 5. Scattering intensity drop of clotrimazole colloids is indicative of colloid disruption even when stabilized by trastuzumab. Scattering intensity after acidification indicates that fulvestrant colloids were stable at both pH values over 6 hours while clotrimazole was only stable at pH 7.

FIG. 24: (A) Trastuzumab-stabilized clotrimazole colloids, (B) but not IgG-stabilized colloids, were internalized by HER2-overexpressing SKOV-3 cells. Colloids labeled with cholesteryl BODIPY dye, cell membrane labelled with wheat germ agglutinin and nuclei labeled with Hoechst.

FIG. 25: CACs were measured in pH-adjusted phosphate buffered saline (PBS) by measuring the minimum concentration at which aggregates begin to form using dynamic light scattering. The CAC of A) an imidazole-containing sorafenib analogue is increased with reducing pH whereas the CAC of B) the parent molecule sorafenib is not pH-dependent (n=3, mean±95% CI).

FIG. 26: (A) Graphically depicts the colloid radius as a function of trastuzumab concentration for trastuzimab-stablized colloids of imidazole-containing sorafenib analogue, (B) shows results for colloid preparation in different pH conditions, with or without the trastuzimab stabilizer. The colloids were prepared in different pH conditions, spun down, extracted from the supernatant by organic solvent, dried, reconstituted in DMSO and measured for absorbance.

FIG. 27: Characterization of the cytotoxicity of sorafenib and its acid-responsive analogue (Sorfenib-AL) against cancer cell lines. Cells were incubated for 3 d in media containing different concentrations of drug, and then their viability was assessed by Presto Blue. IC50's were calculated from the resulting dose-response data using GraphPad Prism 7 (n=3, mean±SD).

FIG. 28: Synthetic scheme depicting the synthesis of acid-responsive fulvestrant analogues bearing different tertiary amines. Reagents and conditions: a) dihydropyran (3 equiv), TFA, DCM, rt 3 d; b) PhI(OAc)₂ (3 equiv), ammonium carbamate (4 equiv), methanol, rt, 4 h; c) bromoacetyl bromide, DCM, 0° C.→rt, 30 min; d) nitrogen nucleophile (NuH), or its hydrochloride salt (NuH×HCl) and DIPEA (5-20 equiv nucleophile), DCM, rt, 3-5 h; e) 2% TFA (>10 equiv) in methanol, rt, 16 h.

FIG. 29. Synthetic scheme depicting the synthesis of acid-responsive fulvestrant analogues bearing different primary amines. Reagents and conditions: a) TBSCl (3 equiv), imidazole (10 equiv), DMF, rt, 16 h; b) PhI(OAc)₂ (3 equiv), ammonium carbamate (4 equiv), methanol, rt, 4 h; c) N-Boc amino acid (glycine or valine) (1.2 equiv), HCTU (1.2 equiv), DIPEA (3.6 equiv), DMF, 0° C.→rt, 16 h; d) acetic acid (5 equiv), TBAF (5 equiv), THF, 60° C., 4 h; e) DCM (78% v/v), TFA (20% v/v), water (2% v/v), 0° C., 15 min, rt, 2 h.

FIG. 30. Mass spectrum of an intermediate in the synthesis of acid-responsive fulvestrant analogues demonstrating a major peak and the target mass-to-charge ratio.

FIG. 31. Mass spectrum of an intermediate in the synthesis of acid-responsive fulvestrant analogues demonstrating a major peak and the target mass-to-charge ratio.

FIG. 32. Mass spectrum of an intermediate in the synthesis of acid-responsive fulvestrant analogues demonstrating a major peak and the target mass-to-charge ratio.

FIG. 33. ¹H NMR spectrum characterizing an acid-responsive fulvestrant analogue bearing a tertiary amine based on dimethylamine.

FIG. 34. ¹³C NMR spectrum characterizing an acid-responsive fulvestrant analogue bearing a tertiary amine based on dimethylamine.

FIG. 35. ESI+ mass spectrum characterizing an acid-responsive fulvestrant analogue bearing a tertiary amine based on dimethylamine.

FIG. 36. ¹H NMR spectrum characterizing an acid-responsive fulvestrant analogue bearing a tertiary amine based on imidazole.

FIG. 37. ¹³C NMR spectrum characterizing an acid-responsive fulvestrant analogue bearing a tertiary amine based on imidazole.

FIG. 38. ESI+ mass spectrum characterizing an acid-responsive fulvestrant analogue bearing a tertiary amine based on imidazole.

FIG. 39. ¹H NMR spectrum characterizing an acid-responsive fulvestrant analogue bearing a tertiary amine based on morpholine.

FIG. 40. ¹³C NMR spectrum characterizing an acid-responsive fulvestrant analogue bearing a tertiary amine based on morpholine (DMF used as an internal standard for quantification).

FIG. 41. ESI+ mass spectrum characterizing an acid-responsive fulvestrant analogue bearing a tertiary amine based on morpholine.

FIG. 42. ¹H NMR spectrum characterizing an acid-responsive fulvestrant analogue bearing a primary amine based on glycine.

FIG. 43. ESI+ mass spectrum characterizing an acid-responsive fulvestrant analogue bearing a primary amine based on glycine.

FIG. 44. ESI+ mass spectrum characterizing an acid-responsive fulvestrant analogue bearing a primary amine based on valine.

FIG. 45: Summary of the chemical structures of acid-responsive fulvestrant analogues and measurements of their critical aggregation concentrations (CACs) over a range of pH values. CACs were measured in pH-adjusted phosphate buffered saline (PBS) by measuring the minimum concentration at which aggregates begin to form using dynamic light scattering. The CACs of acid-responsive fulvestrant analogues are increased with reducing pH whereas the CAC of the parent molecule fulvestrant is not pH-dependent (n=3-8, mean±SEM).

FIG. 46: CACs were measured in pH-adjusted phosphate buffered saline (PBS) by measuring the minimum concentration at which aggregates begin to form using dynamic light scattering. The CAC of siramesine increases with reducing pH (n=4, mean±95% CI).

FIG. 47: Formulation of siramesine with ultrapure polysorbate 80 (UP80) or bovine serum albumin (BSA) results in stable colloids. Siramesine colloids (500 μM) were formulated with either UP80 (A) or BSA (B) in phosphate buffered saline. Hydrodynamic diameter was measured over time and remained relatively constant.

FIG. 48: Resuspension of lyophilized siramesine colloids (5 mg/mL) stabilized with bovine serum albumin (BSA, 50 mg/mL) results in relatively monodisperse colloids at high concentrations, as measured by DLS. (A) Size and PDI values indicate the presence of a single population of nanometer-sized colloidal drug aggregates. (B) Scattering intensity measurements indicate that stabilizing excipients enable the resuspension of siramesine colloids after lyophilization.

FIG. 49: Formulation of sorafenib with aDT as an excipient results in stable colloids. Sorafenib colloids (200 μM) was formulated with 10 or 100 μg/mL of aDT in phosphate buffered saline (PBS). (A) Hydrodynamic diameter and (B) scattering intensity were measured by dynamic light scattering (DLS) at multiple time points and remained relatively constant over 48 hours.

FIG. 50: Acid-responsive attenuated diphtheria toxin (aDT) stabilizing excipient confers enhanced cytotoxicity of sorafenib colloids to a cancer cell line. SK-OV-3 cells were incubated for 1 h with bovine serum albumin (BSA; 100 μg/mL), aDT (100 μg/mL), BSA-stabilized sorafenib collodis (100 μg/mL BSA, 200 μM sorafenib) or aDT-stabilized sorafenib colloids (100 μg/mL aDT, 200 μM sorafenib) in complete culture media. Cell metabolic activity was determined after 3 days by Presto Blue cell viability reagent and normalized to cells without treatment. aDT-stabilized sorafenib colloids result in the greatest reduction in cell viability.

Table 1: Colloid-forming drugs with pKa's>5 show pH-dependent critical aggregation concentrations (CACs) between physiological pH 7.4 and endosomal pH 5.5. CACs were measured in pH-adjusted phosphate buffered saline (PBS) by dynamic light scattering (n=3, mean±95% CI) and pKa's were obtained from DrugBank.ca.

Table 2: Description of each variable and function used in calculation of the critical aggregation concentration (CAC). This calculation objectively applies a common approach for measuring the CAC where assays are used in which colloidal drug aggregates produce a signal and soluble drug molecules do not. For example, the light scattering intensity of a drug solution does not increase with increasing drug concentration until the CAC is reached, when colloidal particles begin to form. By increasing the concentration above the CAC, the number of colloids increases somewhat linearly, and the scattering intensity increases similarly. This behavior is reflected in Equation S4, which describes a line, the slope of which transitions from zero to positive once the CAC is reached.

Table 3: Plots of scattering intensity versus concentration that are used to calculate the CAC of each drug at pH 7.4 and 5.5.

Table 4: Formulations relating treatment name to final concentrations of each component. For imaging and flow cytometry experiments, CholEsteryl BODIPY 542/563 C11 was added before colloid formation to a final concentration of 500 nM.

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

As used herein, the term “in need thereof” is used to refer to a judgment made by a physician or other caregiver (e.g., a veterinarian) that a subject requires or will benefit from treatment or preventative care. This judgment is made based on a variety of factors that are in the realm of the physician's or caregiver's expertise.

As used herein, “pharmaceutically acceptable” means approved or approvable by a regulatory agency of a federal or a state/provincial government or the corresponding agency in countries other than the United States or Canada, or that is listed in the U.S. Pharmacopoeia or other generally recognized pharmacopoeia for use in animals, including in humans.

As used herein, the term “prodrug” will be understood to refer to a compound that, following administration to a subject, is metabolized into a pharmacologically active drug.

As used herein, the term “subject” refers to a human or a non-human animal (e.g., a mammal).

The present application provides a colloidal drug aggregate, and related composition, that is designed to enhance the effect of colloid-bound drug against its molecular target by being responsive to a change in environment as stimulus to trigger drug release from the stable colloids. The change in environment occurs without adding an exogenous reagent by exploiting an endogenous stimulus, such as, for example, the acidic environment of the stomach or of the endosomes and lysosomes of cells. Alternatively, in order to demonstrate this behaviour in the absence of a living organism, the colloidal drug aggregate composition can be triggered to release drug by the addition of an exogenous trigger, such as, for example, an acidic agent or solution.

The formation of some colloidal drug aggregates is pH-dependent, with the colloidal form of weakly basic drugs dissolving at low pH.¹⁵⁻¹⁷ This behaviour allows colloids that are stable outside the cell to be disrupted intracellularly by the acidic endo-lysosomal pathway.¹⁸⁻²¹ Colloidal drug aggregates stabilized with a targeting antibody have been shown to enter cancer cells via endocytosis,²² such that acidification within the cells can trigger release.

The present application provides a stable acid-responsive composition comprising a colloidal aggregate comprising one or more drugs, which facilitates acid-triggered release of the drug(s) following administration. Optionally, the stable colloidal aggregate is a targeted composition. The present application further provides methods of manufacture and use of such colloidal drug aggregates. Use of the stable, targeted colloidal drug aggregate-containing composition described herein results in an enhanced effect of the colloid-bound drug against its molecular target over non-colloidal controls and non-acid responsive colloidal controls, due to, for example, colloid endocytosis and subsequent endosomal escape.

Colloidal Drug Aggregates

Many drugs form colloidal drug aggregates in biologically relevant environments, including pharmaceutical excipients, cell culture media and simulated gastrointestinal fluids. Colloidal aggregates have unique nanostructures and are quite different from some other self-assembled drug nanoparticles such as nanocrystals. Colloids typically have a diameter in the range of from about 50 to about 1000 nm and form spontaneously by phase separation on addition to a liquid, such as water or an aqueous solution (the continuous phase). Aggregation is concentration dependent; at low concentrations the compound is fully solubilized and as the concentration increases to a “critical aggregation concentration” (or “CAC”) the compound spontaneously self-assembles, or aggregates, as a colloid. When the colloid is diluted, the colloid aggregate spontaneously disassembles and the compound returns to its solubilized form. The CAC of a compound is determined by a combination of intrinsic properties of the compound and extrinsic properties, such as, for example, temperature, nature of the continuous phase, and salt concentration. Also, the presence of solubilizing or stabilizing excipients can affect the CAC of a compound.

Non-limiting examples of drugs that form colloidal drug aggregates include clotrimazole, econazole, miconazole, sulconazole, intraconazole, celecoxib, mefenamic acid, oxaprozin, candesartan cilexetil, manidipine, nicardinpine, cinnarizine, glyburide, amiodarone, etravirine, delvirdine, nelfinavir, chlorpromazine, clofazimine, clopidogral sulfate, fenofibrate, meclizine, menatetrenone, pranlukast, raloxifene, triclabendazole, methylene blue, bexarotene, lapatinib, crizotinib, fulvestrant, nilotinib, sorafenib, vemurafenib, gefitinib, imatinib, pazopanib, cabozantinib, and siramesine.

TABLE
Non-limiting examples of drug compounds known to form
colloidal drug aggregates

| DRUG | INDICATION/MECHANISM |
|---|---|
| Clotrimazole | anti-fungal |
| Econazole | anti-fungal |
| Miconazole | anti-fungal |
| Sulconazole | anti-fungal |
| Intraconazole | anti-fungal |
| Celecoxib | NSAID |
| Mefenamic Acid | NSAID |
| Oxaprozin | NSAID |
| Candesartan cilexetil | Hypertension |
| Manidipine | Hypertension |
| Nicardinpine | Hypertension, Ca-channel inhibitor |
| Cinnarizine | calcium channel blocker |
| Glyburide | diabetes, K-channel inhibitor |
| Amiodarone | anti-arrhythmic |
| Etravirine | anti-HIV, non-nucleoside reverse |
| | transcriptase inihibtor |
| Delvirdine | anti-HIV, non-nucleoside reverse |
| | transcriptase inihibtor |
| Nelfinavir | Antiretroviral, Anti-HIV |
| Chlorpromazine | anti-psychotic |
| Clofazimine | anti-bacterial |
| clopidogral sulfate | anti-platelet/anti-dotting agent |
| Fenofibrate | hypercholesterolemia |
| Meclizine | Anti-histimine |
| Menatetrenone | Hemostatic agent |
| Pranlukast | anti-asthmatic |
| Raloxifene | selective estrogen receptor modulator |
| Triclabendazole | anti-parasitic |
| Methylene Blue | |
| Congo Red | |
| Evans Blue | |
| Bexarotene | cutaneous T cell lymphoma |
| Lapatinib | HER2 inihibtor, breast cancer |
| Crizotinib | ALK inhibitor, non-small cell lung cancer |
| Fulvestrant | ER antagonist, breast cancer |
| Nilotinib | Bcr-Abl inhibitor, chronic myelogenous leukemia |
| Sorafenib | multi-TM, renal cell carcinoma, hepatocellular |
| | carcinoma, thyroid carcinoma |
| Vemurafenib | B-Raf inhibitor, melanoma |
| Gefitinib | EGFR inhibitor, non-small cell lung cancer |
| Imatinib | ABL, c-kit inhibitor, chronic myelogenous leukemia |
| Pazopanib | multi-kinase inhibitor (VEGFR, PDGFR), renal cell |
| | carcinoma |
| Cabozantinib | c-Met and VEGFR2 inhibitor, thyroid cancer, |
| | renal cell carcinoma |
| Siramesine | Sigma receptor agonist |

In accordance with some embodiments, the colloidal drug aggregate composition of the present application comprises an ionizable drug. For example, the colloidal composition is formed from a colloidal aggregation of one or more ionizable drugs. At least one of the one or more ionizable drugs is basic, or weakly basic, such that the colloid is susceptible to dissolution in the presence of an acid or acidic environment. In particular, the protonated form of the ionizable drug in the colloidal formulation has a pKa of at least 4.0. Preferably, the pKa of the protonated form of the ionizable drug is at least 4.5, or at least 5.0, or at least 6, or at 6.5. Most dissolved drug molecules are positively charged at pH values below this pKa and uncharged at pH values greater than this pKa.

Suitable drugs for use in colloid drug aggregates as taught herein are drugs that solubilize in acidic environments and are colloids under physiologically neutral (e.g., approximately pH 7.4—slightly basic) conditions. An “acidic environment” is defined herein as an environment having a pH of less than 7.4. By ionizing at acidic pH, the drugs in colloids become soluble. The drug colloid aggregate comprising one or more ionizable drug is acid responsive, in that the colloid aggregate will dissolve or disassemble in upon contact with acid or introduction into an acidic environment, thereby releasing the drug from the colloid aggregate. The pH of the acid or acidic environment at which dissolution or disassembly occurs depends on the pKa of the ionizable drug in its protonated form.

Non-limiting examples of ionizable drugs that can be included in the colloidal drug aggregate composition of the present application include lapatinib, clotrimazole, nilotinib, pazopanib, and siramesine.

Drugs that form colloidal drug aggregates but that form colloids that do not respond to acid (e.g., sorafenib and fulvestrant), can be chemically modified by addition of an ionizable functional group to form an analogue that is responsive to acid. These drugs are either non-ionizable, or do not ionize in the presence of pH less than 4, until they have been chemically modified to include such an ionizable functional group. These drugs retain activity following chemical modification to include the ionizable functional group, or are metabolized in vivo to become biologically/pharmaceutically active. Accordingly, in accordance with some embodiments, the colloidal drug aggregate composition of the present application comprises an ionizable drug analogue that is responsive to acid.

Drug analogues useful in the formation of the colloidal drug aggregates as described herein, are drug molecules that have been chemically modified to include an ionizable functional group and that, as a result of the chemical modification, have a protonated form that has a pKa of at least 4.0. Preferably, the pKa of the protonated form of the ionizable drug analogue is at least 4.5, or at least 5.0, or at least 6, or at 6.5.

Suitable drug analogues for use in colloid drug aggregates as taught herein are drug analogues that solubilize in acidic environments and are colloids under physiologically neutral (e.g., approximately pH 7.4—slightly basic) conditions. By ionizing at acidic pH, the drug analogues in colloids become soluble. The drug colloid aggregate comprising one or more ionizable drug analogue is acid responsive, in that the colloid aggregate will dissolve or disassemble in upon contact with acid or introduction into an acidic environment, thereby releasing the drug analogue from the colloid aggregate. The pH of the acid or acidic environment at which dissolution or disassembly occurs depends on the pKa of the ionizable drug in its protonated form.

Non-limiting examples of suitable ionizable functional groups include amines, such as, primary amines, secondary amines, tertiary amines, imines, heterocyclic amines, aromatic amines. More specific examples of suitable amines include, hydroxylamine, hydrazine, imidazole, amine, and triazoles. Alternatively, an aggregator drug can be modified by chemical conjugation to an amine-containing molecule to form an analogue that is responsive to acid. Suitable amine-containing molecules include an amino nitrogen atom which is bound to three other atoms, at least one of which attaches the nitrogen atom to the aggregator molecule through a series of covalent bonds. Optionally, this amino nitrogen atom may be a part of one or more natural amino acids or derivatives thereof, including, but not limited to, β-alanine, N,N-dimethylglycine, N,N-dimethyl-β-alanine, histidine, serine, threonine, asparginine, glutamine, proline, alanine, leucine, isoleucine, methionine, phenylalanine, tyrosine, tryptophan, glycine, or valine. In this embodiment, the drug analogue exhibits similar activity to the unmodified drug, or the drug analogue functions as a prodrug that is metabolized in vivo to form an active drug molecule.

Non-limiting examples of drug analogues that can be included in the colloidal drug aggregate composition of the present application include a sorafenib analogue (e.g., an imidazole-containing sorafenib analogue), and a fulvestrant analogue (e.g., a glycine or valine modified fulvestrant, or a fulvestrant analogue bearing an amine derived from dimethylamine, imidazole, or morpholine).

The colloidal formulation additionally comprises a stabilizing agent or excipient. Most colloid-forming drugs aggregate at micromolar or sub-micromolar concentrations. Colloids formed without stabilizing excipients are often polydisperse and precipitate over several hours in physiological media, are unstable and, therefore, unsuitable for use according to the present invention. Excipients, including polymers, proteins, and other colloid-forming compounds, such as other drugs or azo-dyes, can control the size and stability of colloidal drug aggregates. In addition to stabilizing colloidal drug aggregates, proteins (e.g., antibodies) can also confer functionality to promote selective uptake by target cells.

In certain embodiments, the colloidal drug aggregate of the present application comprises a corona. As used herein, the term “corona” refers a surface-bound layer of adsorbed proteins and/or other stabilizing excipient(s), onto a colloidal aggregate, for example, resulting from the exposure of the aggregate to solution and media containing proteins and/or other stabilizing excipient(s). In one embodiment, in which the conditions for concentration and nature of the proteins and/or excipient(s) in the solution and media is known, the corona may be well defined and characterized. The corona formation and its characteristics may be influenced by various factors such as the surface chemistry of the nanoparticle, the colloidal aggregate size, and the composition of the continuous phase.

Proteins that can be used for the protein corona may be, but are not limited to, albumins, immunoglobulins, caseins, insulins, hemoglobins, lysozymes, α-2-macroglobulin, fibronectins, vitronectins, fibrinogens, lipases, transferrin, apolipoproteins, bacterial toxins, and the like. Proteins, peptides, enzymes, antibodies and combinations thereof, may also be used to stabilize the colloidal aggregates. Accodring to an embodiment, the use of novel antibodies or FDA-approved antibodies such as toclizumab, natalizumab, vedolizumab, alemtuzumab, cetuximab, daratumumab, ofatumumab, panitumumab, pertuzumab, trastuzumab, dinutuximab, obinutuzumab, ramucirumab, ipilumumab, nivolumab, pembrolizumab, brentuximab, catumaxomab, basilixumab, ibritomumab may be tailored to stabilize the colloidal aggregates. The target/indication for these FDA approved antibodies are provided in the table below.

TABLE
Non-limiting examples of antibody therapeutics
that have been approved by FDA

| ANTIBODY | TARGET/INDICATION |
|---|---|
| Toclizumab | IL-6R, rheumatoid arthritis |
| Natalizumab | a4-integrin, Crohn's disease, multiple scelrosis |
| Vedolizumab | a4b7-integrin, Crohn's disease, ulcerative colitis |
| Alemtuzumab | anti-CD52, chronic lymphocytic leukemia, cutaneous |
| | T-cell lymphoma, multiple scelrosis |
| Ceniximab | EGFR, colorectal cancer, head and neck cancer |
| daratumumab | CD38, multiple myeloma |
| ofatumumab | CD20, chronic lymphocytic leukemia |
| Panitumumab | EGFR, colorectal cancer |
| Pertuzumab | HER2, breast cancer |
| Trastuzumab | HER2, breast cancer, |
| Dinutuximab | GD2, neuroblastoma |
| obinutuzumab | CD20, chronic lymphocytic leukemia |
| Ramucirumab | VEGFR2, non-small cell lung cancer, |
| | gastric/gastroesophageal junction adenocarcinoma |
| Ipilumumab | CTLA-4, melanoma |
| Nivolumab | PD- 1 , melanoma |
| Pembrolizumab | PD-1, melanoma, NSCLC, |
| Brentuximab | CD20, ADC with monomethyl auristatin E for |
| | chronic lymphocytic leukemia |
| Catumaxomab | anti-EpCAM/anti-CD3, malignant ascites |
| Basilixumab | IL-2R alpha chain, immunosupression |
| Ibritomumab | CD20, conjugated to radioisotope chelator |
| | for non-hodgkin's lymphoma |

In accordance with other embodiments, the acid-responsive colloidal drug composition comprises a combination of one or more non-ionizable, or ioniziable, drugs with an acid-responsive stabilizer. This acid-responsive stabilizer acts to disrupt the colloid or cellular membranes in an acidic environment. In particular, an acid-responsive stabilizing agent is one that undergoes a morphological and/or functional change when the pH is reduced to less than about 6.5, or less than about 6, or less than about 5.5, or less than about 5, or less than about 4.5, or less than about 4.

Non-limiting examples of acid-responsive stabilizers include bacterial toxins, such as attenuated diphtheria toxin or variants thereof.

Accordingly, certain embodiments of the present application provide colloidal drug aggregate compositions that are useful in targeting a drug for treatment of a disease or disorder in a subject. In specific embodiments, the disease or disorder is selected from the diseases and disorders associated with the listed FDA-approved antibodies, as set out in the table above. This list is not exhaustive; therapeutic antibodies and targeting antibodies continue to be developed and can be incorporated in the colloidal drug aggregate compositions of the present application as a stabilizing agent, a targeting agent, or both.

In other embodiments, the colloidal drug aggregate composition of the present application comprises a stabilizing agent that is another aggregator drug, which may or may not be acid responsive. In a particular embodiment, the colloidal drug aggregate composition comprises an acid responsive (i.e., ionizable) drug and a non-acid responsive drug. In one example of this embodiment, the acid responsive drug is lapatinib and the non-acid responsive drug is fulvestrant.

Colloidal drug aggregate formulations of the present application can be formulated simply by dissolving a drug or drug analogue in a water-miscible solvent (e.g., DMSO) and adding water, or an aqueous solution, to the dissolved drug and allowing the spontaneous formation of colloids. One or more stabilizing agents are incorporated into either aqueous or organic phase prior to colloid formation, or to the mixture after colloid formation. For convenience, the formation of the colloidal drug aggregate is often done at ambient temperature, typically without the need for further mixing or stirring. The amount of the drug used will depend on the CAC for that drug under the formulation conditions used. Colloids are usually detectable within seconds of mixing the drug, or drug analogue, in the water-miscible solvent with the aqueous phase. In accordance with certain embodiments in which the colloidal drug aggregate formulation is for pharmaceutical use, the one or more stabilizing agents are cytocompatible. Also, as noted above, in certain embodiments the one or more stabilizing agents are acid-responsive.

In some instances, the pH of the colloid solution needs to be adjusted to facilitate colloid formation or for colloid stability. The pH can be adjusted using an adjusting agent that is an acid (e.g., HCl or citric acid) or a base (e.g. NaOH), as necessary. pH adjustment is typically performed by adding the pH adjusting agent as the final formulation step. Depending on the amount of pH adjusting agent required, it may be necessary to reduce the amount of water, or aqueous solution, used in the colloid formation.

Pharmaceutical Formulations and Use Thereof

The present application further provides pharmaceutical formulations for administration of a colloidal drug aggregate composition to a subject in need thereof. The pharmaceutical formulation of the present application comprises a colloidal drug aggregate, optionally in combination with a pharmaceutically acceptable carrier, diluent, excipient or medium, wherein the colloidal drug aggregate comprises one or more drugs and a stabilizing agent, and wherein the colloidal aggregate disrupts, dissolves or disassembles when the acid-responsive composition is in an acid environment.

In certain embodiments, the pharmaceutical composition comprises a combination of two or more colloidal drug aggregate compositions.

Suitable pharmaceutical compositions of the present application will generally include an amount of the one or more drugs to give a range of final concentrations, depending on the intended use.

In certain embodiments, the pharmaceutical compositions of the present application comprise a colloidal drug aggregate composition as the sole active component, alone or in combination with one or more desired pharmaceutically inactive additives, excipients, and/or components (e.g., polymers, fillers, carriers, excipients, diluents, disintegrating additives, lubricants, solvents, dispersants, absorption promoting additives, controlled release additives, anti-microbial additives, preservatives, sweetening additives, colorants, flavors, dyes, or the like), and no other active pharmaceutical ingredient(s).

In other embodiments, the pharmaceutical composition comprises a combination of two or more colloidal drug aggregate compositions as the active components, alone or in combination with one or more desired pharmaceutically inactive components as listed above.

In alternative embodiments, the pharmaceutical composition comprises one or more colloidal drug aggregate compositions in combination with one or more other therapeutic agent.

Accordingly, the present application further comprises a method of providing drug therapy to a subject by administering a pharmaceutical composition as described above. Such a method is particularly useful for delivering or targeting the one or more drugs in the pharmaceutical composition to an acidic target site of the patient (e.g., stomach, lysosomes endosomes, etc.).

In a specific embodiment, the pharmaceutical composition comprises a colloidal drug aggregate composition comprising lapatinib. Accordingly, the present application further comprises a method of treating cancer, such as breast cancer, comprising administration of a colloidal drug aggregate composition comprising lapatinib to a subject in need thereof. Optionally, the method additionally comprises administration of another chemotherapeutic agent before, after or simultaneous with administration of the colloidal drug aggregate composition comprising lapatinib.

In another specific embodiment, the pharmaceutical composition comprises a colloidal drug aggregate composition comprising lapatinib and fulvestrant. Accordingly, the present application further comprises a method of treating cancer, such as breast cancer, comprising administration of a colloidal drug aggregate composition comprising lapatinib and fulvestrant to a subject in need thereof. Optionally, the method additionally comprises administration of another chemotherapeutic agent before, after or simultaneous with administration of the colloidal drug aggregate composition comprising lapatinib and fulvestrant.

In another specific embodiment, the pharmaceutical composition comprises a colloidal drug aggregate composition comprising clotrimazole. Accordingly, the present application further comprises a method of treating a fungal infection, such as a fungal skin infection, comprising administration of a colloidal drug aggregate composition comprising clotrimazole to a subject in need thereof. Optionally, the method additionally comprises administration of another chemotherapeutic agent before, after or simultaneous with administration of the colloidal drug aggregate composition comprising clotrimazole.

In another specific embodiment, the pharmaceutical composition comprises a colloidal drug aggregate composition comprising sorafenib or an analogue of sorafenib. Accordingly, the present application further comprises a method of treating cancer, such as kidney cancer, liver cancer, or thyroid cancer, comprising administration of a colloidal drug aggregate composition comprising the sorafenib analogue to a subject in need thereof. Optionally, the method additionally comprises administration of another chemotherapeutic agent before, after or simultaneous with administration of the colloidal drug aggregate composition comprising the sorafenib analogue.

In another specific embodiment, the pharmaceutical composition comprises a colloidal drug aggregate composition comprising an analogue of fulvestrant. Accordingly, the present application further comprises a method of treating cancer, such as breast cancer, comprising administration of a colloidal drug aggregate composition comprising the fulvestrant analogue to a subject in need thereof. Optionally, the method additionally comprises administration of another chemotherapeutic agent before, after or simultaneous with administration of the colloidal drug aggregate composition comprising the fulvestrant analogue.

To gain a better understanding of the invention described herein, the following examples are set forth. It should be understood that these examples are for illustrative purposes only. Therefore, they should not limit the scope of this invention in any way.

### EXAMPLES

### Example 1: Colloidal Lapatinib Aggregate Preparation and Use

Methods

Materials

Lapatinib and sorafenib were purchased from MedChemExpress. Nilotinib and pazopanib were purchased from Cedarlane. Fulvestrant was purchased from SelleckChem. Poly(D,L-lactide-co-2-methyl-2-carboxy-trimethylene carbonate)-graft-poly(ethylene glycol) (PLAC-PEG) was synthesized by ring-opening polymerization using a pyrenebutanol initiator to a molecular weight of 12 kDa and conjugated with an average of three 10 kDa PEG chains/backbone as previously described.⁴³ Ultrapure polysorbate 80 (UP80) was purchased from NOF America Corporation. Clotrimazole, norethindrone, Dimethyl sulfoxide (DMSO), transferrin, EDTA, dodecane, lecithin, polyacrylic acid (PAA), methylcelluose, and hydroxypropylmethylcellulose (HPMC) were purchased from Sigma-Aldrich. Transferrin-Alexa Fluor 488 conjugate, RPMI 1640 cell culture medium, penicillin-streptomycin solution, trypsin-EDTA solution, Hank's balanced salt solution, PrestoBlue cell viability reagent, CholEsteryl BODIPY™ 542/563 C11, CellMask Green™ 1000× solution, and 7-AAD were purchased from Thermo Fisher Scientific. Fetal bovine serum and Dulbecco's phosphate buffered saline were purchased from Wisent Bio Products. Ultrapure Congo red was purchased from Enzo Life Sciences. HPLC grade acetonitrile and methanol were purchased from Caledon Laboratories. Mass spectrometry grade formic acid was purchased from Fluka.

Calculation of the Critical Aggregation Concentration (CAC)

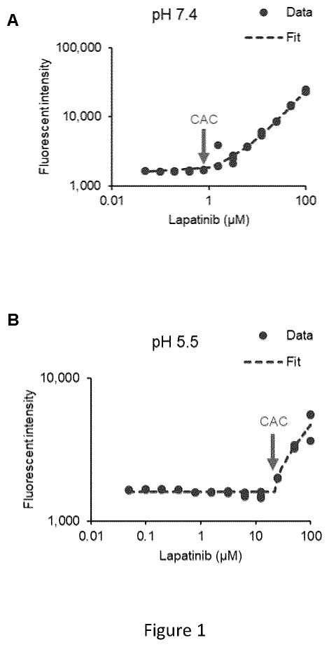

CAC values are calculated by preparing colloidal formulations with different concentrations and measuring the scattering intensity (or fluorescence intensity when appropriate). The data is then fit to Equation S4, which was derived from first principles (Equations S1-S3), using the MATLAB curve fitting toolbox to obtain the critical aggregation concentration (CAC) under the conditions used. Table 2 describes the meaning of each variable; FIG. 1 shows the result of curve-fitting this model to experimental data.

$$Signal = {\sum }_{i} {Signal}_{i} = {Signal}_{BL} + {Signal}_{colloids} \qquad S1$$

### Assuming Constant Colloid Diameter

$$\begin{matrix}{Signal}_{colloids} ∝ {C}_{agg , colloids} & S2 \\ {C}_{agg , colloids} = \{ \begin{matrix}0 , & {C}_{agg} < {CAC}_{agg} \\ {C}_{agg} - {CAC}_{agg} , & {C}_{agg} \geq {CAC}_{agg}\end{matrix} & S 3\end{matrix} = H ( {C}_{agg} - {CAC}_{agg} ) \cdot ( {C}_{agg} - {CAC}_{agg} )$$

### Substituting and Adding a Qualitative Correction Factor for Diameter Variations

Signal=Signal_{BL}+m+H(C_{agg}−CAC_{agg})·(C_{agy}−CAC_{agg})

Colloidal Drug Aggregate Formulation

Colloidal drug aggregates were formulated as described previously.⁸ Briefly, the colloids formed spontaneously when water was added to drug that was dissolved in a water-miscible organic solvent (usually DMSO). Final colloid suspensions were typically made at a 1 mL scale with 1% (v/v) final DMSO concentration. First, solutions of drug, polymer, and dye were prepared in 10 μL DMSO at 100×the final concentration. For colloids formulated under serum-free conditions, 890 μL of double distilled water was then added, followed by 100 μL of 10×PBS (for experiments without cells) or 10×RPMI 1640 (for cell experiments). For colloids formulated under 10% (v/v) serum conditions, 800 μL of double distilled water was added, followed by 90 μL of 10×PBS or 10×media, and finally 100 μL of FBS. Transferrin stabilized colloids were prepared by supplementing the water added with a small amount of 5 mg mL⁻¹ transferrin in PBS. When the pH of the colloid solution needed to be adjusted, the amount of water added was reduced by 10 μL, and 10 μL of aqueous citric acid was added as the last formulation step. The concentration of citric acid used depended on the desired final pH. In PBS, the concentration was 0.12 M for pH 6.5, 0.3 M for pH 5.5, 0.35 M for pH 5.2, and 0.5 M for pH 4.5. In PBS with 10% (v/v) FBS, the concentration was 0.15 M for pH 6.5, 0.35 M for pH 5.5, and 0.6 M for pH 4.5. For time-based studies, the colloid suspensions were incubated at 37° C. between time points.

Characterization by Dynamic Light Scattering

Colloid diameter, polydispersity index (dispersity), and scattering intensity were measured by dynamic light scattering (DLS) using a DynaPro Plate Reader II (Wyatt Technologies) that was optimized by the manufacturer for detection of colloidal aggregates (i.e. 100-1000 nm particles). The instrument was configured with a 60 mW 830 nM laser and detector angle of 158°. A 100 μL sample of each formulation was pipetted into a 96-well plate and measured with 20 acquisitions per sample.

Characterization of Zeta Potential

Colloids were prepared as described above, but with a single addition of 0.1 mM KCl solution instead of water and buffer. Zeta potential was immediately assessed using a Malvern Nano-ZS (Malvern Panalytical).

Fluorexcent Intensity Characterization

Colloid suspensions were prepared as described above. A 100 μL sample of each formulation was pipetted into a 96 well plate. Fluorescence was measured using a Tecan Infinite Pro 200 plate reader.

Characterization by Transmission Electron Microscopy (TEM)

Colloidal formulations (5 μL) were deposited onto freshly glow-discharged 400 mesh carbon coated copper TEM grids (Ted Pella, Inc.) and allowed to adhere for 5 min. Excess liquid was removed, followed by a quick wash with 5 μL water. Grids were then imaged on a LEO 912B Energy Filtered TEM operating at 120 kV.

Assessment of Colloid-Bound Transferrin

Colloid formulations were prepared as described above with transferrin-Alexa Fluor 488 conjugate as the stabilizer. At predetermined time points during incubation at 37° C., formulations were centrifuged at 16,000×g for 1 h, followed by withdrawal of 100 μL, of supernatant for measurement. The amount of transferrin remaining in the supernatant was measured using fluorimetry (λₑₓ=488 nm, kₑₘ=530 nm). The fraction of transferrin bound to the colloid was calculated with Equation 1.

$${f}_{bound} = 1 - {f}_{free} = 1 - \frac{{I}_{colloid + Tf 488}}{{I}_{Tf 488}} \qquad 1$$

Assessment of Drug Release

Drug release was measured by centrifuging to pellet the colloids, and then by quantifying the drug in the supernatant. Colloid formulations were centrifuged at 16,000×g for 1 h, followed by withdrawal of 100 μL for quantification. Another 100 μL was withdrawn for DLS analysis to confirm that the colloids had completely settled out of solution. Non-centrifuged colloid suspensions were used as controls.

Membrane Permeability Assay

Supported artificial lipid membranes were prepared according to the manufacturer instructions (EMD Millipore cat #MATRNPS50). Briefly, a 1% (w/w) solution of egg lecithin in dodecane was prepared by sonicating until the solution was no longer cloudy. A 5 μL aliquot of this phospholipid solution was dropped by pipette into each well of the mesh plate, which wet the membrane and caused it to become translucent. Receiver solution was prepared as 1% (v/v) DMSO and 10% (v/v) FBS in PBS and 340 μL was pipetted into each well of the receiver plate. Lapatinib and fulvestrant solutions ranging from 0 to 5 μM for constructing the standard curve were prepared in identical media and pipetted into separate wells of the receiver plate. Colloids were prepared in PBS containing 10% (v/v) FBS as described above, and 150 μL was pipetted into the mesh plate wells corresponding to the receiver solution in the receiver plate. The mesh plate and receiver plate were gently mated, resulting in the receiver solution wetting the bottom of the phospholipid membrane. A plate cover was added, and the assembly edges sealed with parafilm to prevent evaporation. The sealed assembly was then incubated at 37° C. for 6 h. Finally, the stack was carefully disassembled and a 200 μL sample of receiver and standard curve solutions was withdrawn for drug quantification as described below. Each donor and receiver well were tested with a pH strip to verify integrity of the membrane. A separate test with Trypan blue verified that the membranes were impermeable to this colloidal dye.

Drug Concentration Quantification

Drug concentration in samples from drug release and membrane permeability assays was quantified by high pressure liquid chromatography coupled with tandem mass spectrometry (HPLC-MS-MS). Protein was precipitated from samples in serum containing media by spiking with 10 μL of formic acid and adding acetonitrile to a final volume of 1 mL. The precipitated samples were then centrifuged at 16,000×g for 5 min to pellet the proteins. The drug-containing supernatant was then diluted in methanol such that the final drug concentration was less than 100 ng mL⁻¹. During the final dilution, internal standards (nilotinib for lapatinib and norethindrone for fulvestrant) were added to a final concentration of 25 ng/mL each. Standard curves were prepared in a similar way, by diluting 100 mM solutions of drug in DMSO with methanol to final concentrations of 100, 75, 50, 25, 10, 5, 2.5, and 1 ng mL⁻¹, each with 25 ng mL⁻¹ of internal standard.

Cell Culture

MDA-MB-231-H2N cells were a generous gift from Dr. Robert Kerbel (Sunnybrook Research Institute, Toronto, ON, Canada). Cells were maintained in a humidified incubator at 37° C. with 5% atmospheric CO₂. Cells were grown in 75 cm² tissue culture flasks with 10 mL RPMI 1640 supplemented with 10% FBS, 10 UI/mL penicillin, and 10 μg mL⁻¹ streptomycin. Cells were passaged twice per week with typical subculture ratio of 1:16.

Cell Viability Experiments

After passaging, cell suspensions were diluted into fresh media and 200 μL was pipetted into each well of a 96 well plate. Two thousand cells per well were plated and allowed to adhere overnight. Then, the media was withdrawn and replaced with treatment formulations (prepared as described above). Cells were incubated during the experiment in a humidified incubator at 37° C. with 5% atmospheric CO₂. For treatments lasting less than 3 d, the treatment solutions were removed after the prescribed time, cells were washed with fresh media, and 200 μL of fresh media was added. Cell viability was assessed 3 days after commencing treatment using the PrestoBlue™ viability assay according to the manufacturer's protocol. Cell viability was reported as a percentage of the vehicle (DMSO with no drug or excipient) control.

Confocal Imaging of Treated Cells

Cells were seeded at approximately 2×10⁵ cells per well in 8-chamber tissue culture treated glass cover slips and allowed to adhere overnight. Treatments were prepared as described above and 300 μL applied to each well. Cells were incubated during the experiment in a humidified incubator at 37° C. with 5% (v/v) atmospheric CO₂. The treatment solutions were removed after the prescribed time, cells washed with fresh media, and 300 μL of fresh media added. For fixed cells, 4% (w/w) aqueous paraformaldehyde was applied for 10 min, followed by staining (when appropriate), and finally blank PBS. For live cell conditions, cells were washed with fresh media, stained, and imaged under Hank's balanced salt solution. Cells were imaged on an Olympus FV1000 inverted confocal microscope using a 1.42 N.A. 60× oil immersion lens (Olympus PLAPON 60XO). Laser and detector settings were held constant between different treatment conditions.

Flow Cytometry

Cells were seeded at approximately 2×10⁵ cells per well in 24-well plates and allowed to adhere overnight. Treatments were prepared as described above and 500 μL was applied to each well. Cells were incubated during the experiment in a humidified incubator at 37° C. with 5% (v/v) atmospheric CO₂. For treatments lasting less than 3 h, the treatment solutions were removed after the prescribed time, cells were washed with fresh media, and 500 μL of fresh media was added. Three hours after treatment initiation, cells were washed three times with media, detached using 500 μL of accutase solution, spiked with 500 μL of media, then centrifuged at 400×g to pellet the cells. Cells were then resuspended in cold flow buffer (PBS supplemented with 2% (v/v) FBS and 2 mM EDTA) and kept on ice until measurement. The flow buffer was supplemented with 7-AAD at 2 μg mL⁻¹ as a vital stain, except for blank controls and in the endosome escape assay where 7-AAD had already been added. Cell fluorescence was quantified using a BD Accuri™ C6 flow cytometer with excitation wavelength of 488 nm and emission filters of 585/40 nm (BODIPY colloid dye) and >670 nm (7-AAD). Data were analyzed using the BD Accuri™ C6 Plus software and reported as the fluorescence of the live cell fraction (gated using scattering and 7-AAD) averaged between three biological replicates.

Results

This study was initiated by identifying colloid-forming drugs that could respond to acidic conditions by measuring the critical aggregation concentration (CAC) of several aggregators as a function of pH (Table 1). Critical aggregation concentrations were calculated by plotting the scattering intensity (indicative of colloid number and size) from dynamic light scattering (DLS) versus concentration (Table 2, FIG. 1, Table 3).^{1, 23} Weakly basic aggregators, such as clotrimazole and lapatinib, had pH-dependent CACs, which enable colloid disruption as a function of pH; conversely, aggregators with pKa values well below the identified pH range, such as fulvestrant and sorafenib, did not dissolve on acidification.

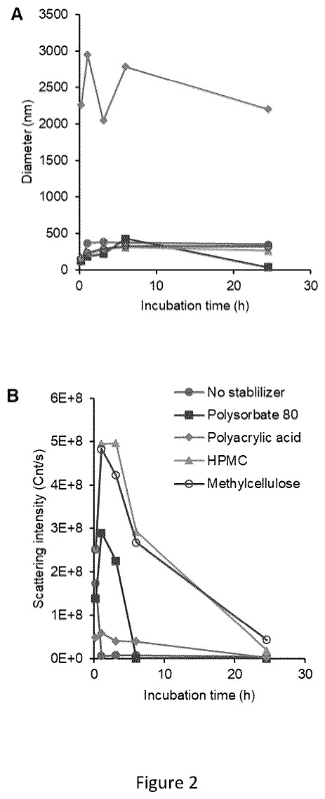

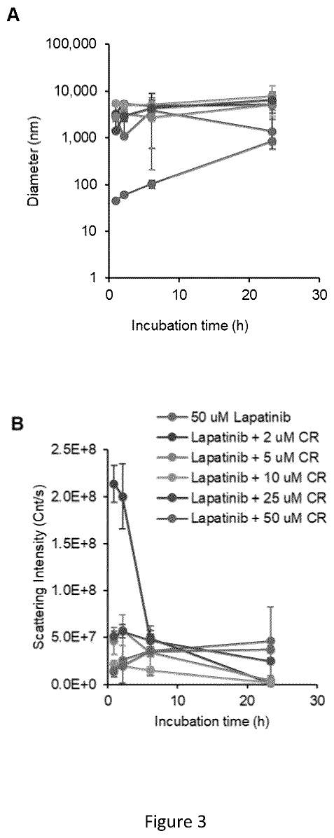

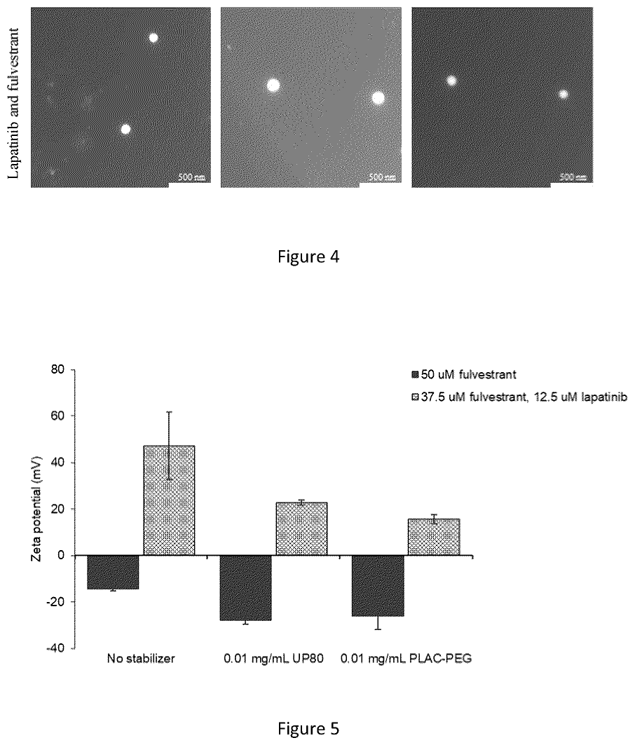

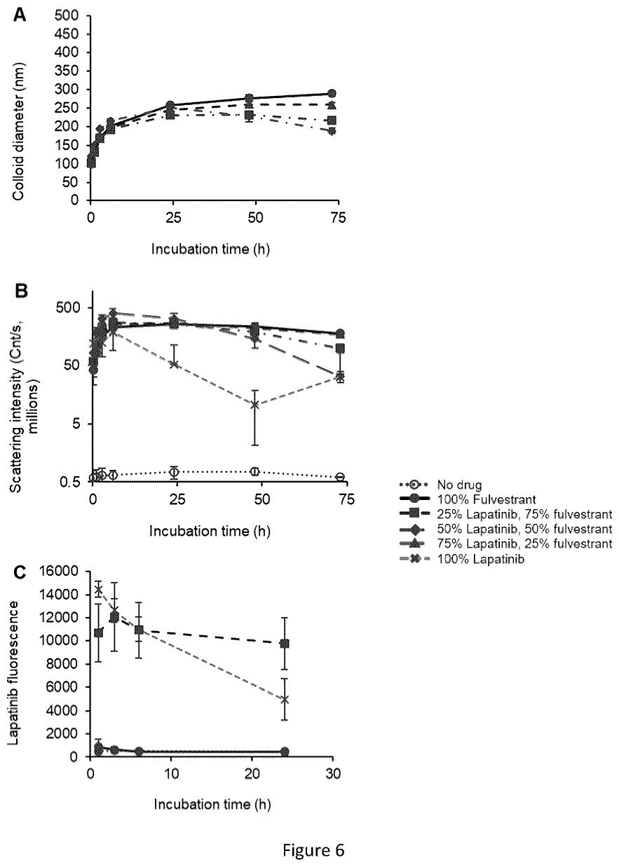

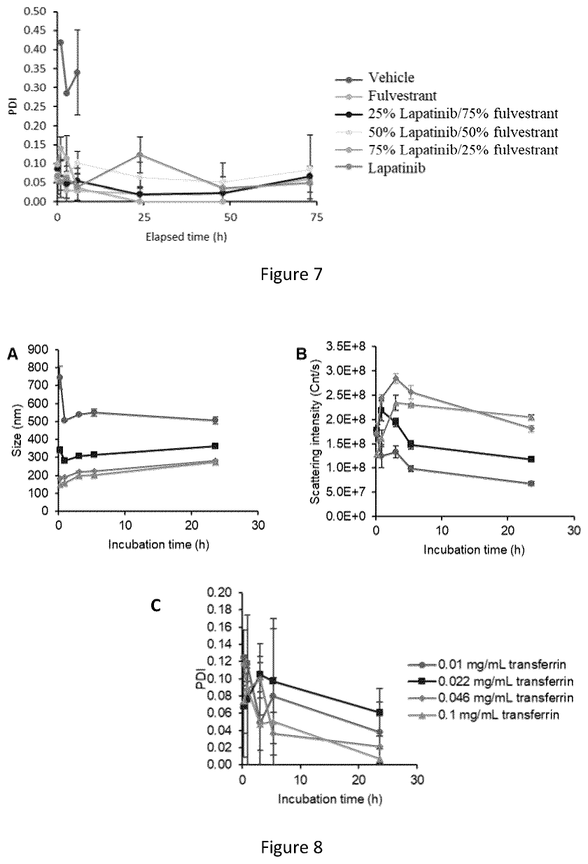

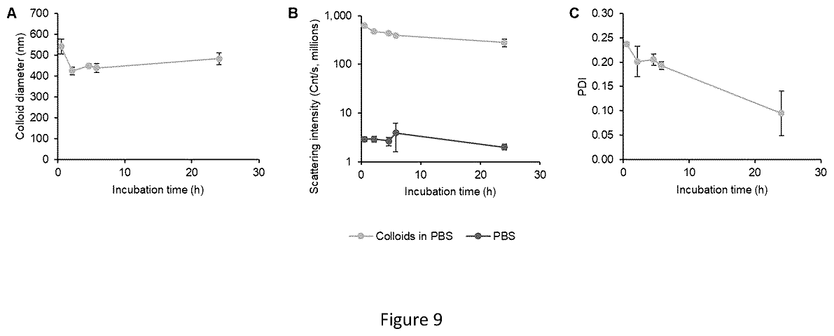

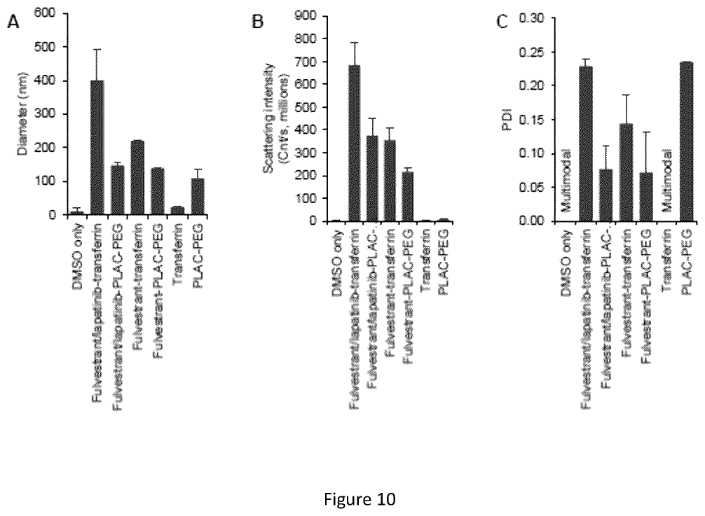

A stable colloidal formulation of lapatinib was developed due to its potency and robust response to acidic pH. Although lapatinib forms colloidal aggregates on its own, they are only transiently stable and precipitate within a few hours (FIG. 2). Excipients were screened to stabilize colloidal lapatinib against precipitation (FIG. 2, FIG. 3)^{8, 24-26} and it was found that co-aggregation with fulvestrant, another colloid-forming drug, resulted in stable, spherical colloids, approximately 200 nm diameter (FIG. 4) with slight positive charge (FIG. 5). It was also found that a 1:3 molar ratio of lapatinib:fulvestrant was best stabilized against precipitation by excipients such as the graft copolymer poly(D,L-lactide-co-2-methyl-2-carboxytrimethylene carbonate)-graft-poly(ethylene glycol) (PLAC-PEG, FIG. 6, FIG. 7) and the protein transferrin (FIG. 8, FIG. 9). It was also confirmed that these colloids were stable in cell culture media (FIG. 10).

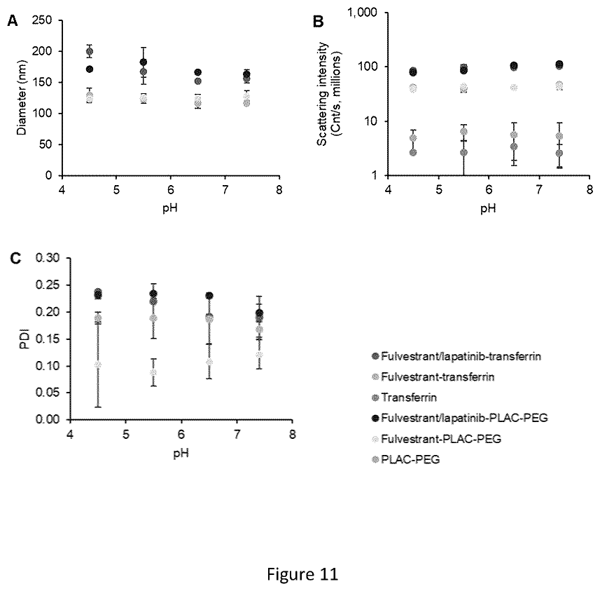

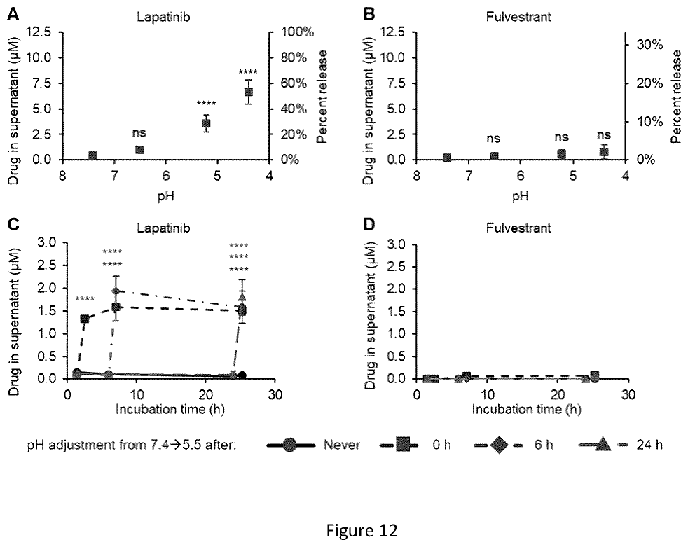

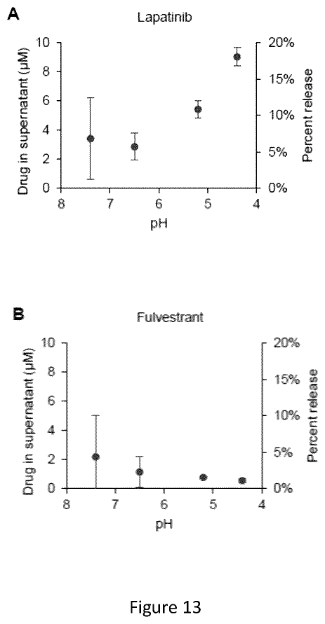

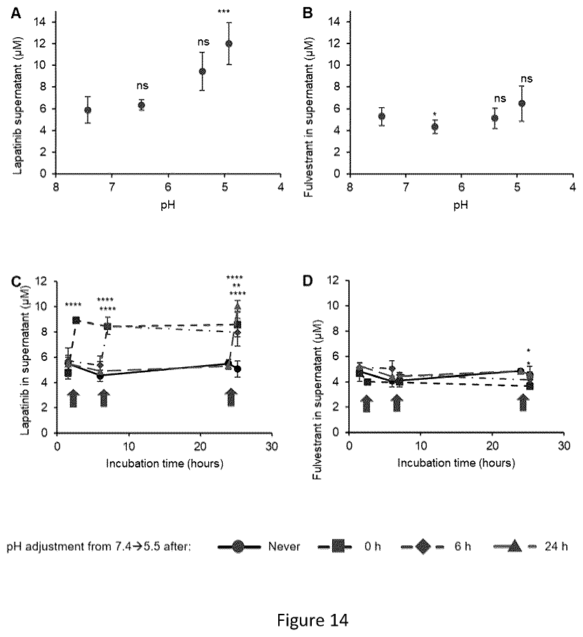

The response of the stable colloids to acidic conditions was investigated. Colloids were formulated in PBS and subsequently acidified, mimicking the pH within the endo-lysosomal pathway. To determine whether the lapatinib-fulvestrant co-colloids changed as a function of pH, colloid size and scattering intensity were measured by DLS (FIG. 11). Relatively little change in colloid size after acidification was observed. Next, the suspension was centrifuged to pellet the colloids, and then free drug in the supernatant was quantified after ensuring that no colloids were present. The amount of released lapatinib increased with increasing acidity (FIG. 12A) whereas the release of the non-ionizable fulvestrant was unaffected by pH (FIG. 12B). Importantly, lapatinib release could be triggered at any time after formulation, and rapidly reached an equilibrium that was sustained for 24 h (FIG. 12C) whereas fulvestrant remained unresponsive to acid (FIG. 12D). The amount of drug released from the colloids remained similar from both 50 μM to 200 μM colloidal aggregates (FIG. 13). Additionally, similar trends were observed (FIG. 14) in PBS containing 10% (v/v) fetal bovine serum (FBS), demonstrating a similar response in serum-containing media. An increased baseline level of drug release in serum-containing media was also observed, which is consistent with previous reports that show how proteins increase the CAC of colloidal aggregators.^{10, 18} These results demonstrate that the stable lapatinib colloids will respond to the acid stimulus in the endo-lysosomal pathway.

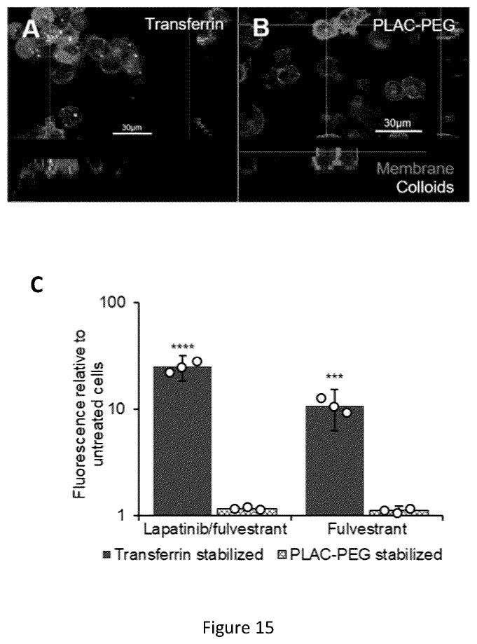

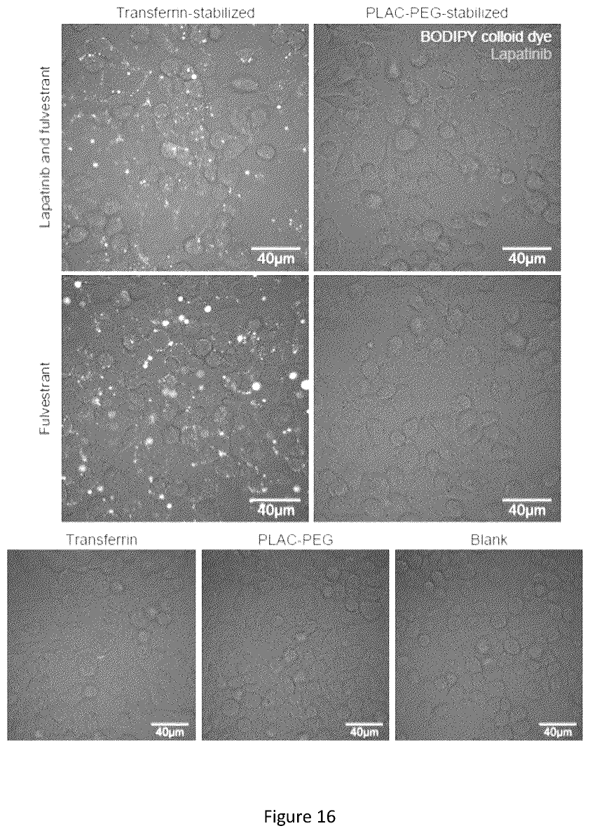

The endocytosis of the stable, acid-responsive colloidal drug aggregates was investigated. To measure uptake, a hydrophobically-modified BODIPY dye, which is fluorescent only when incorporated within the colloid, ²² was incorporated. These colloids appeared as punctate structures within MDA-MB-231-H2N cells (FIG. 15, FIG. 16). Although minimal uptake of the PLAC-PEG stabilized colloids observed, it was found that transferrin-stabilized colloids were taken up to a much greater extent (FIG. 3C). This result held for both lapatinib/fulvestrant colloids and colloids of fulvestrant alone, and aligns with previous research demonstrating that stabilizers that interact specifically with cells can elicit endocytosis.²²

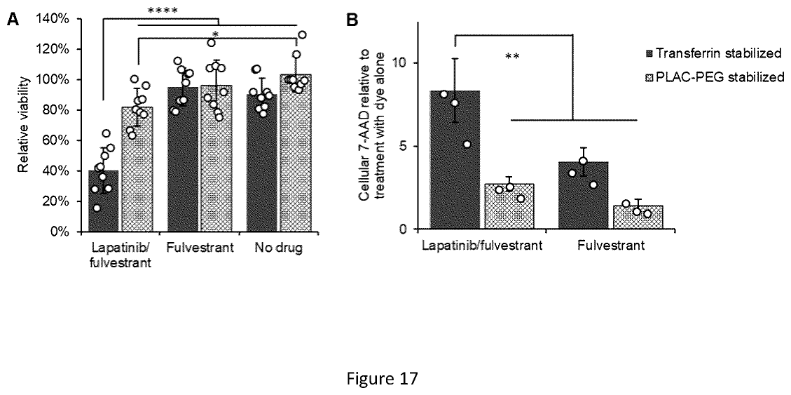

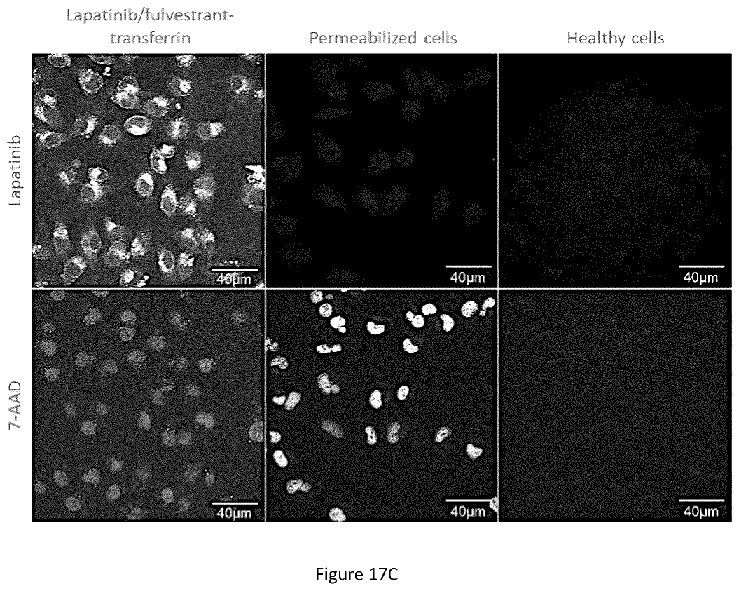

The in vitro toxicity of acid-responsive, stable lapatinib/fulvestrant colloids was then examined against lapatinib-sensitive, HER2-overexpressing MDA-MB-231-H2N cells. The transferrin-stabilized lapatinib/fulvestrant colloids were substantially more toxic than any other formulation, including PLAC-PEG stabilized lapatinib/fulvestrant colloids that were minimally endocytosed (FIG. 17A).

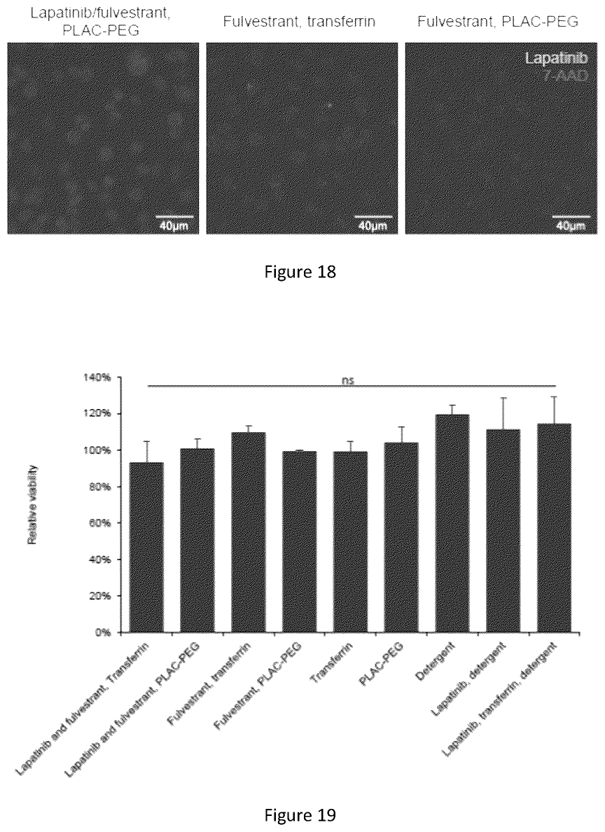

To determine the mechanism of the increased toxicity of endocytosed lapatinib-containing colloids versus non-endocytosed colloids, the ability of colloidal lapatinib to disrupt the endosomes was investigated using a fluorescence dequenching assay.^{29, 30} Incorporating the membrane impermeant nuclear stain, 7-aminoactinomycin D (7-AAD), into the colloids allowed measurement of its fluorescence in MDA-MB-231-H2N cells. The fluorescence of 7-AAD intensifies on DNA binding. Therefore, 7-AAD staining should only be observed in the nucleus following both colloid endocytosis and subsequent endosomal escape. Lapatinib/fulvestrant-transferrin colloids resulted in higher nuclear 7-AAD fluorescence than fulvestrant-transferrin colloids and PLAC-PEG stabilized formulations (FIG. 17B-C, FIG. 18). Viability of the cells remained unchanged after 3 h of treatment, indicating that drug toxicity did not interfere with this assay (FIG. 19).

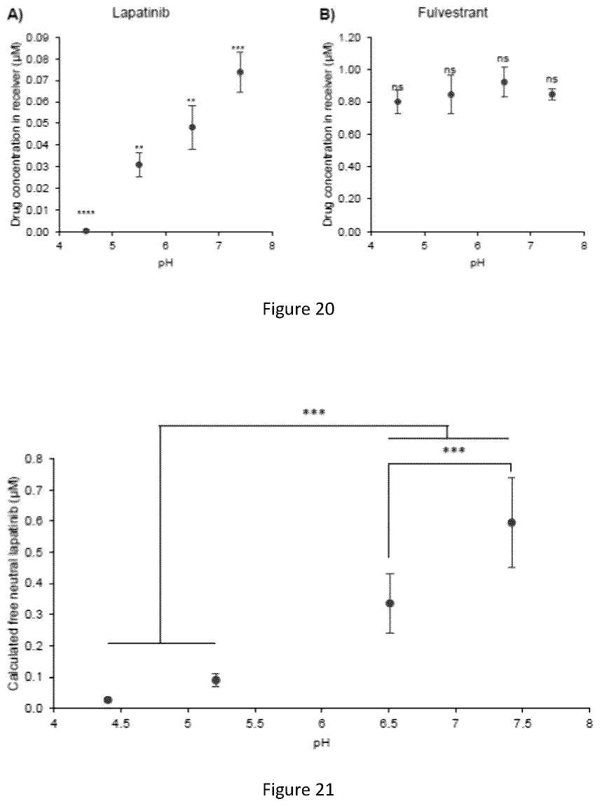

An alternative explanation to endosome disruption is simple diffusion of lapatinib from the endosomes, which was probed with a membrane permeability assay. The transmembrane diffusion of lapatinib was reduced when the colloids were acidified (FIG. 20), which, without wishing to be bound by theory, was likely because the concentration of free, uncharged lapatinib decreased with increasing acidity (FIG. 21).

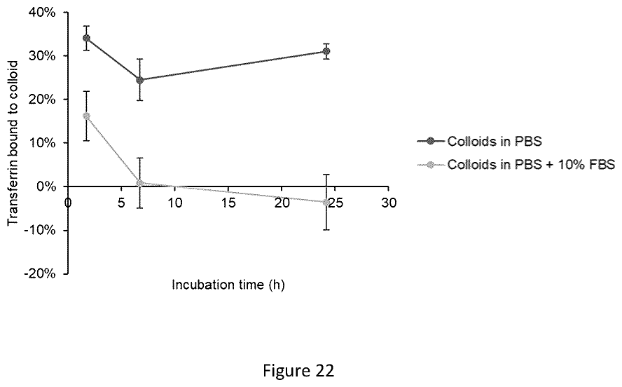

Notwithstanding the importance of transferrin for cellular uptake, its stability in serum was investigated since displacement of the macromolecular coronas by serum proteins can affect circulating nanoparticles.²⁷ To test whether serum proteins could displace the transferrin stabilizer, colloidal drug aggregates stabilized with fluorophore-labelled transferrin were formulated and then pelleted by centrifugation. Quantifying the fraction of transferrin remaining in the supernatant, it was found that most of the transferrin was displaced after 6 hours of incubation in 10% serum (FIG. 22).

Discussion

This study demonstrates that it is possible to trigger the release of weakly basic colloid-forming drugs from stable colloidal drug aggregates by acidification of the medium. By exploiting the local acidity in the endo-lysosomal pathway, drug release and its consequent cytotoxicity was triggered, thereby overcoming a key challenge of colloidal stability, and hence inactivity, after cell uptake. Thus, whereas previous research has shown that colloidal aggregation can cause false negative hits in cytotoxicity assays,^{11, 12} this effect can be overcome by protonation of the colloidal drug and subsequent en-dosome disruption.

Interestingly, it was found that the amount of lapatinib released was less than its CAC at a comparable pH. Without wishing to be bound by theory, this behaviour may be attributed to the fulvestrant, which remains in the colloidal state and acts as a sink for lapatinib. This hypothesis is supported by studies on the phase behaviour of co-colloids that show a reduction in effective CAC when multiple drugs co-exist in the colloid.³¹⁻³³ This physical interaction between lapatinib and fulvestrant suggests that they mix to form co-colloids and may explain how fulvestrant stabilized lapatinib against precipitation.

It was found that endocytosis of the acid-responsive lapatinib-fulvestrant colloids greatly enhanced their cytotoxicity due to increased drug transport from the endosomes into the cytosol. This endosomal escape could occur by one of two mechanisms: either the increased concentration of free lapatinib enhances the amount of lapatinib diffusion across the endosomal membrane or the weakly basic lapatinib disrupts the integrity of the endosomes through osmotic pressure effects.^{29, 34, 35} As lapatinib is slow to cross membranes under acidic conditions, the enhanced diffusion mechanism is unlikely. Transport of ionizable drugs, such as protonated (cationic) lapatinib at reduced pH, diffuse more slowly through the lipid membranes.^{36, 37} This prediction was supported by the estimate of free, uncharged lapatinib as a function of pH. With respect to the proton-sponge mechanism, the 7-AAD fluorescence quenching assay showed that acid-responsive colloidal drug aggregates can disrupt endosomes, leading to drug leakage into the cytosol and ultimately enhancing drug cytotoxicity. This type of endosomal escape strategy has been exploited to deliver therapeutics that are unable to escape the endosomes themselves.^{35, 38, 39} This approach has been adapted here and has been surprisingly found to effectively deliver a new type of cargo: colloidal drug aggregates. Furthermore, the approach employed here successfully avoided the use of acid-responsive but pharmacologically inert excipients by using a drug that is naturally acid-responsive. Such pharmacologically active drug colloids can be used instead of the traditionally used inert carriers to deliver proteins or nucleic acids.

Notwithstanding these results, colloidal drug aggregates are admittedly in dynamic equilibrium with free drug, and a small amount of that free drug may diffuse across lipid membranes. As a result, cells that are highly sensitive to lapatinib may be killed even in the absence of colloid endocytosis. Another consequence of this behaviour is that drug diffusion out of the endo-lysosomal pathway could slowly occur simultaneously with the proton sponge effect.

The present study demonstrates that endosomal escape of lapatinib is enhanced by the acidic microenvironment. Importantly, endosomal escape in live cells was shown to occur through a membrane disruption mechanism. Furthermore, it was shown that only colloidal drugs that are inherently acid-responsive can have their release triggered by acidic conditions whereas those that are unresponsive to acid cannot.

Conclusions

Recent attempts to exploit colloidal drug aggregates have been, hindered, until now, by the inability to control drug release. The present study showed acid-triggered release from stable colloidal drug aggregates, endosomal disruption, and enhanced cytotoxicity. The selective, stimulus-responsive release of drugs from colloidal aggregates was demonstrated. This provides demonstration of controlled release of a therapeutic molecule from a colloid that is inactive until its target is reached.

### Example 2: Trastuzumab-Stabilized Clotrimazole Colloids

Methods

Materials

Fulvestrant was purchased from Selleck Chemicals. Clotrimazole, IgG from human serum, and RPMI 1640 cell culture media were purchased from Sigma-Aldrich. Trastuzumab was obtained from Roche. CholEsteryl BODIPY 542/563 C11, Hoechst 33342, and wheat germ agglutinin Alexa Fluor 647 conjugate were purchased from Thermo Fisher scientific. SKOV3 cells were purchased from ATCC. Fetal bovine serum was purchase from Wisent Bio Products.

Colloid Formulation and Characterization by Dynamic Light Scattering

Colloidal drug aggregates were formulated as described as described in Example 1 using clotrimazole or fulvestrant in place of lapatinib. In particular, colloids of fulvestrant (non-responsive) or clotrimazole (acid-responsive) were formulated at 50 μM and stabilized with 3 μM trastuzumab at pH 7 followed by acidification of pH 5. These formulations were assessed by dynamic light scattering as described in Example 1.

Cell Culture and Confocal Microscopy

Cell culture was carried out as described in Example 1. SKOV3 cells were seeded at 10,000 per well in 16-well glass chamber slides. Fluorescent clotrimazole colloids were prepared as described above, with the addition of 500 nM CholEsteryl BODIPY 542/563 C11. Trastuzumab or IgG were added to a final concentration of 3.5 μM, the formulations were incubated for 10 minutes, and then FBS was added to a final concentration of 5% (v/v). The cell media was replaced with the colloid formulations and incubated for 3 h. The formulations were removed and the cells were fixed with 4% paraformaldehyde. Following fixation, wheat germ agglutininin Alefa Fluor 647 conjugate was added to stain the cell membranes according to the manufacturer protocol and counterstained with Hoechst. Cells were then imaged on an Olympus FV1000 confocal microscope at 60× magnification.

Results

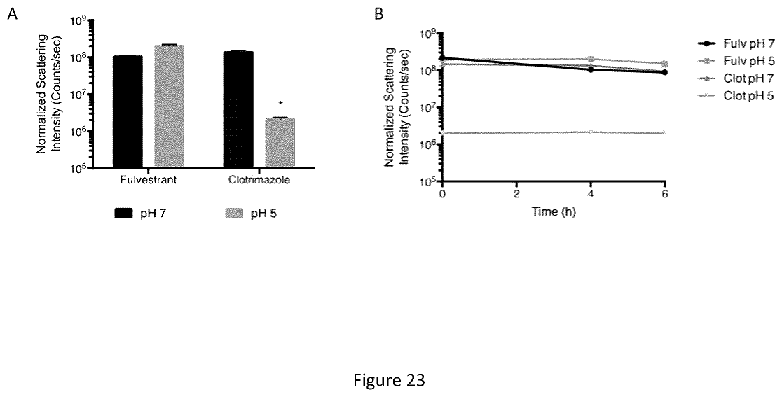

As shown in FIG. 23 scattering intensity after acidification indicates that fulvestrant colloids were stable at both pH values (pH 7 and pH 5) over 6 hours while clotrimazole is only stable at pH 7. This indicates that the clotrimazole-containing colloidal drug aggregate was acid-responsive, while the fulvestrant-containing colloidal drug aggregate was not acid-responsive.

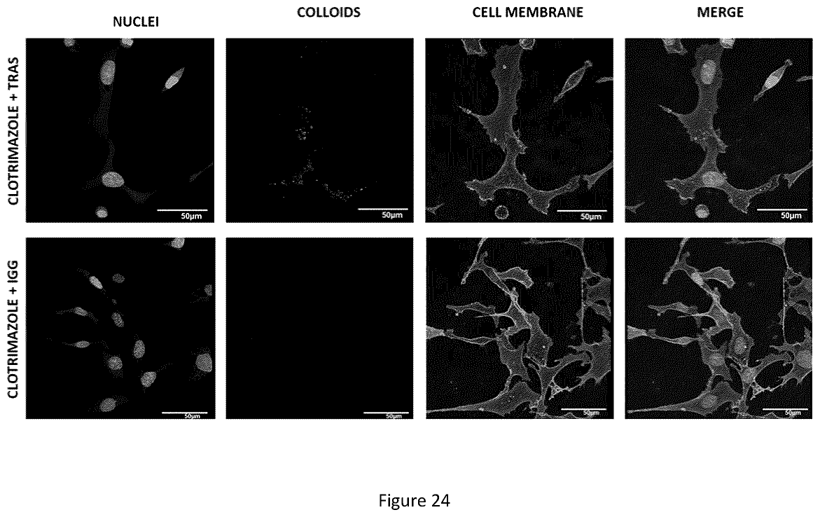

FIG. 24 demonstrates that the trastuzumab-stabilized clotrimazole colloids, but not IgG-stabilized colloids, are internalized by HER2-overexpressing SKOV-3 cells.

Discussion

These results demonstrated that the colloid-forming compound clotrimazole is acid-responsive, and that colloids of clotrimazole can target cancer cells by coating the colloids with targeting antibodies.

### Example 3: Sorafenib Analogue-Containing Colloids

Methods

Materials

A sorafenib analogue containing a pendant imidazole functional group on the N-methylpyridine-2-carboximide was synthesized to specifications by Enamine. Phosphate buffered saline was purchased from Wisent Bio Products. Hydrochloric acid was purchased from VWR. Cell lines were obtained as a generous gift from R. Kerbel (MDA-MB-231-H2N) or from ATCC (BT474, SKBR3, SKOV3).

Colloid Formulation and CAC Measurement

Colloids were formulated as described in Example 1. Briefly, solutions of sorafenib analogue in DMSO were prepared at various concentrations and then diluted 100-fold with PBS at different concentrations. 100 μL of each sample was analyzed by DLS and the CAC's calculated as described in Example 1.

Cell Culture and IC50 Determination

Cell culture and experiments were carried out as described in Example 1. IC50's were calculated using GraphPad Prism 7 from cell viabilities measured after treating with difference concentrations of drug for 3 days.

Results

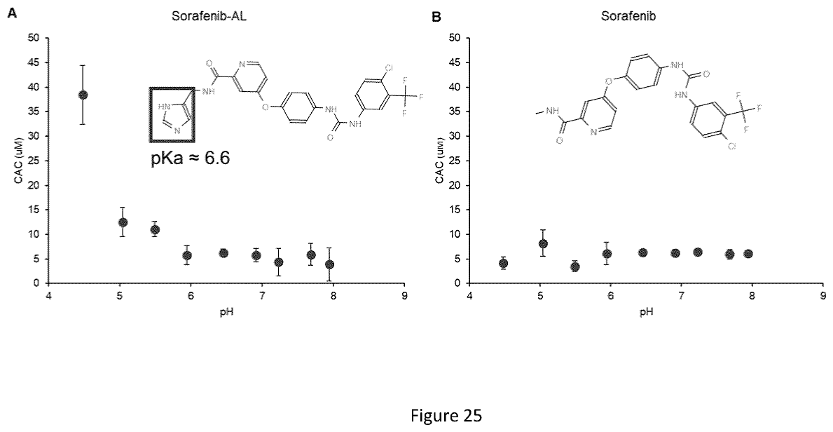

FIG. 25 shows a comparison of the CACs for sorafenib and the sorafenib analogue over a range of pH values. The increase in CAC values found for the sorafenib analogue as the pH decreases is indicative of the acid responsiveness of the colloidal drug aggregate comprising the sorafenib analogue.

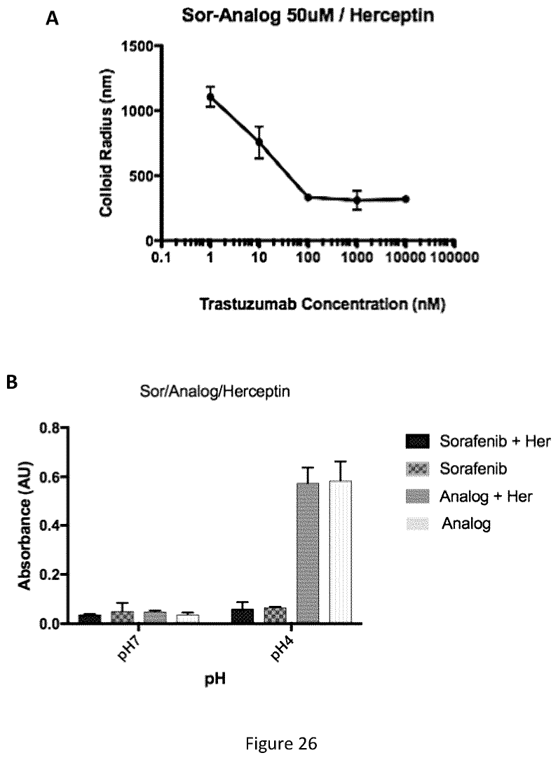

FIG. 26 further demonstrates the acid responsiveness of a colloidal drug aggregate comprising the sorafenib analogue, with or without Herceptin as a stabilizing agent. In contrast, the colloidal drug aggregate comprising sorafenib, with or without Herceptin as a stabilizing agent, did not demonstrate acid responsiveness.

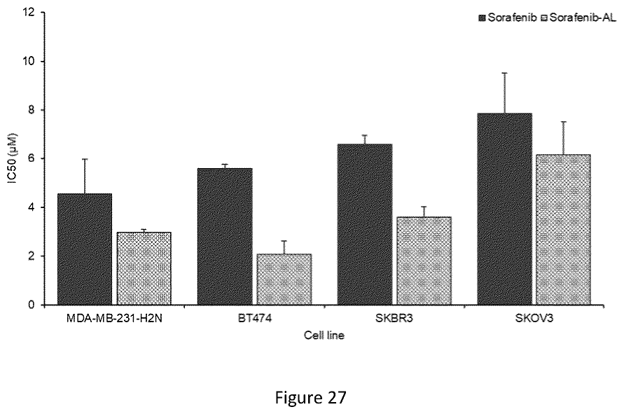

This study also included an investigation of the cytotoxicity of sorafenib in comparison to the cytotoxicity of the sorafenib analogue. As shown in FIG. 27, the analogue exhibited similar (slightly increased) activity against all cancer cell lines tested.

Discussion

These experiments demonstrate that an aggregator (in this case a drug) that does not respond to acid can be made responsive by modification with an ionizable functional group, while retaining the pharmacologic activity of the aggregator.

### Example 4: Fulvestrant Analogue-Containing Colloids

Methods

Materials

Fulvestrant was purchased from MedChemExpress. Dimethyl sulfoxide (DMSO), tert-butyldimethylsilyl chloride (TBSCl), (diacetoxyiodi)benzene, ammonium carbamate, N-Boc-glycine, N-Boc-valine, N,N-diisopropyl-N-ethylamine (DIPEA), tetra-N-butyl ammonium fluoride (TBAF) solution, tetrahydrofuran (THF), trifluoroacetic acid (TFA) bromoacetyl bromide, dimethylamine hydrochloride, and morpholine were purchased from Sigma-Aldrich. Dimethylformamide (DMF) was purchased from Alfa Aesar. Ethyl acetate, tert-butyl methyl ether (MTBE), hexanes, methanol, dichloromethane (DCM), ethanol, and glacial acetic acid were purchased from Caledon. Imidazole was purchased from Bio Basic. HCTU was purchased from AnaSpec. Silica (SilicaFlash P60™) was purchased from SiliCycle.

Synthesis of Ionizable Fulvestrant Analogues Modified with Tertiary Amines

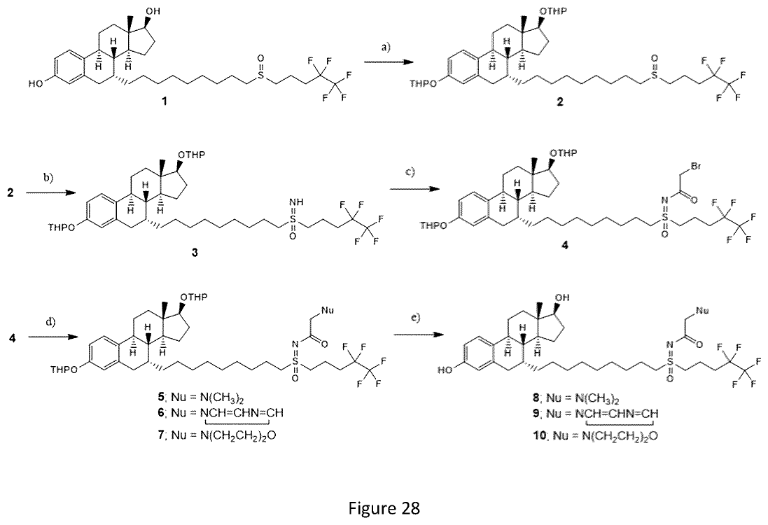

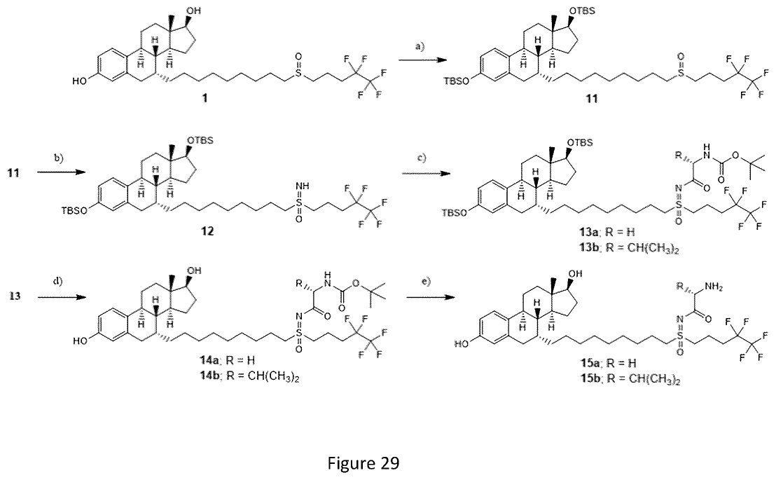

Fulvestrant (species 1 in FIG. 28 and FIG. 29) analogues bearing tertiary amine functional groups were synthesized using a 5-step process summarized by FIG. 28. Reagents and conditions: a) dihydropyran (3 equiv), TFA, DCM, rt 3 d; b) PhI(OAc)₂ (3 equiv), ammonium carbamate (4 equiv), methanol, rt, 4 h; c) bromoacetyl bromide, DCM, 0° C.→rt, 30 min; d) nitrogen nucleophile (NuH), or its hydrochloride salt (NuH×HCl) and DIPEA (5-20 equiv nucleophile), DCM, rt, 3-5 h; e) 2% TFA (>10 equiv) in methanol, rt, 16 h.

3,17β-Bis((tetrahydro-2H-pyran-2-yl)oxy)-7-α-[9-(4,4,5,5,5,-pentafluoropentylsulphinyl) nonyl]estra-1,3,5-(10)-triene (2): 3,4-Dihydro-2H-pyran (248 μL, 2.72 mmol) and trifluoroacetic acid (12.6 μL, 165 μmop were added under stirring to a solution of fulvestrant (500 mg, 824 μmol) in dichloromethane (8 mL). The reaction was stirred at room temperature for 3 d, then extracted (1× sat. NaHCO₃, 1× brine), dried (MgSO₄), and filtered. The solvent was removed under reduced pressure, yielding a clear viscous oil which was used without further purification. R_{f}=0.25 (1:1 hexanes:ethyl acetate).

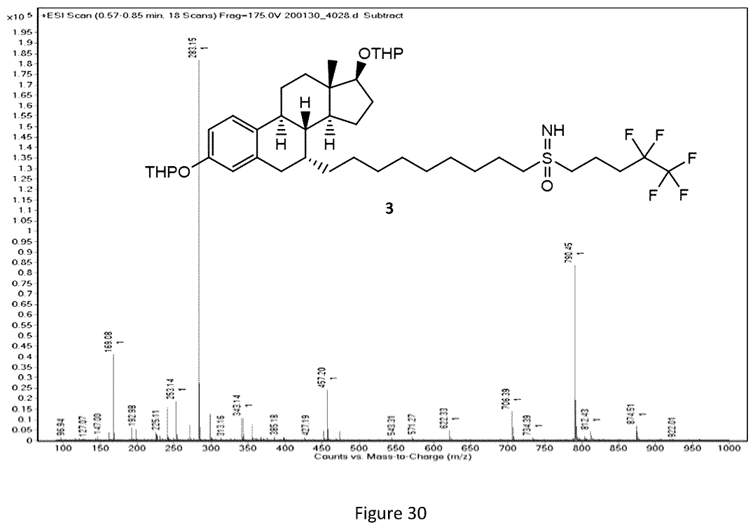

3,17β-Bis((tetrahydro-2H-pyran-2-yl)oxy)-7-α-[9-(4,4,5,5,5,-pentafluoropentyl sulphonimidoyl)nonyl] estra-1,3,5-(10)-triene (3): Finely ground ammonium carbamate (85.2 mg, 1.09 mmol) and (diacetoxyiodo)benzene (221 mg, 685 μmop were added to a stirred solution of crude 2 (192 mg, ≤248 μmot) in methanol (5 mL). After a few minutes, gas (presumably CO₂) was evolved and a yellow color developed. After 4 h, the reaction was diluted in ethyl acetate and extracted (2×PBS, 1×brine), dried (MgSO₄), and filtered. The solvent was removed and the product purified by column chromatography (silica, 1:1 hexanes:ethyl acetate) to give 3 as a clear oil (57.88 mg, 73.26 μmol 30% cumulative yield). R_{f}=0.2 (1:1 hexanes:ethyl acetate); MS (ESI+) m/z [M+H]⁺ calculated for C42H65F5NO₅S: 790.4511, found: 790.4503. FIG. 30 shows the structure of 3 and its mass spectrum.

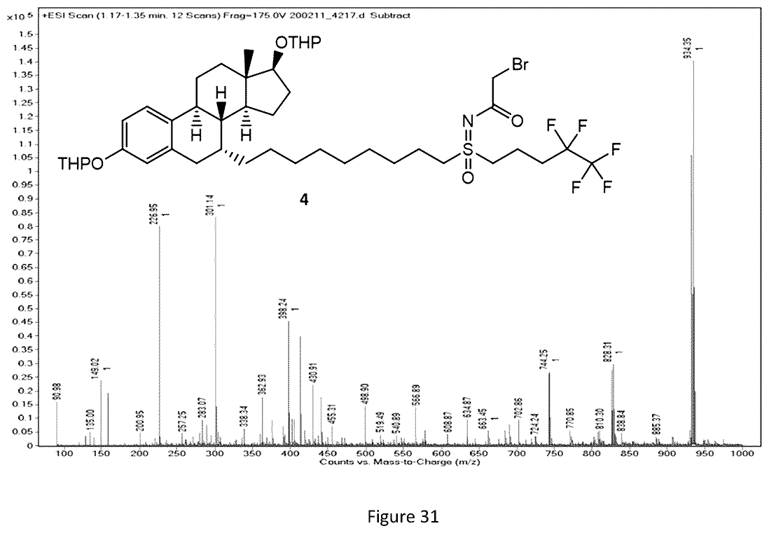

N-(2-Bromoacetyl)-(3,17β-bis((tetrahydro-2H-pyran-2-yl)oxy)-7-α-[9-(4,4,5,5,5,-pentafluoropentylsulphonimidoyl)nonyl]estra-1,3,5-(10)-triene) (4): 3 (57.9 mg, 73.3 μmop was dissolved in dichloromethane (2 mL) and N,N-diisopropylethylamine (63.8 μL, 366 μmol) added. The solution was cooled on ice, and then a solution of bromoacetyl bromide (12.8 μL, 147 μmop in dichloromethane (2 mL) added under agitation. The now amber-coloured solution was allowed to warm to room temperature and stirred for 30 minutes. Then, 100 μL of saturated Na₂CO₃ was added and the solvent removed under air. The residue was then dispersed in ethyl acetate and extracted (3×PBS, 1× brine), dried (MgSO₄), filtered, and evaporated under nitrogen. The crude product was purified by column chromatography (silica, 4:1 hexanes:ethyl acetate) to give 4 as a slightly yellow oil (40.5 mg, 44.1 μmol, 60%). R_{f}=0.4 (4:1 hexanes:ethyl acetate), 0.85 (1:1 hexanes:ethyl acetate); ¹H NMR (500 MHz, CDCl₃) δ 7.17-7.10 (m, 1H), 6.63 (dd, J=8.4, 2.8 Hz, 1H), 6.54 (d, J=3.1 Hz, 1H), 5.05 (dt, J=5.2, 2.7 Hz, 1H), 4.69-4.59 (m, 1H), 4.08-3.96 (m, 2H), 3.96-3.84 (m, 3H), 3.90-3.87 (m, 2H), 3.79-3.69 (m, 2H), 3.66-3.42 (m, 6H), 3.40-3.27 (m, 3H), 2.90-2.82 (m, 1H), 2.70 (dd, J=16.7, 4.8 Hz, 1H), 2.38-1.08 (m, 27H), 1.08-0.85 (m, 6H), 0.83-0.76 (m, 4H); MS (ESI+) m/z [M+Na]⁺ calculated for C₄₄H₆₅BrF₅NNaO₆S: 932.3528, found: 932.3529. FIG. 31 shows the structure of 4 and its mass spectrum.

N-(2-Dimethylaminoacetyl)-(3,17β-bis((tetrahydro-2H-pyran-2-yl)oxy)-7-α-[9-(4,4,5,5,5,-pentafluoropentylsulphonimidoyl)nonyl]estra-1,3,5-(10)-triene) (5): A solution of dimethylamine hydrochloride (285 mg) and DIPEA (70 μL, 400 μmol) in DCM (1.5 mL) was added to a solution of 4 (97 mg, 100 μmol) in 1 mL DCM. The reaction was agitated for 2.5 h, diluted with 10 mL ethyl acetate, and extracted (2× water, 1× brine). The extract was dried (MgSO₄) and the solvent removed under nitrogen. The resulting amber oil was used without further purification.

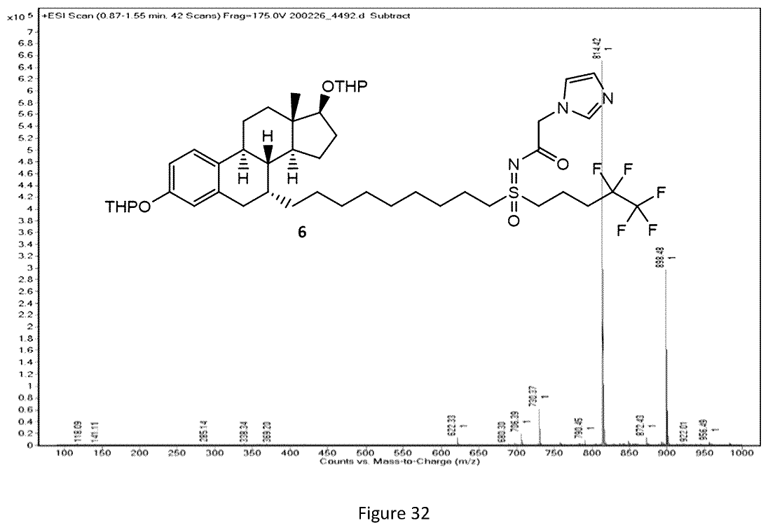

N-(1H-Imidazol-1-ylacetyl)-(3,17β-bis((tetrahydro-2H-pyran-2-yl)oxy)-7-α-[9-(4,4,5,5,5,-pentafluoropentylsulphonimidoyl)nonyl]estra-1,3,5-(10)-triene) (6): A solution of imidazole (16 mg, 0.23 mmol) in dichloromethane (1 mL) was added to 4 (18 mg, 20 μmol), agitated for 3 h, and then the solvent removed under nitrogen. The residue was then resuspended in ethyl acetate and extracted (2× water, 1× brine), dried (MgSO₄), and the solvent removed under nitrogen followed by vacuum. The resulting oil (18 mg, 20 μmol, 99%) was used without further purification. MS (ESI+) m/z [M+H]⁺ calculated for C₄₇H₆₉F₅N₃O₆S: 898.4822, found: 898.4820 ([M+H−THP]+ and [M+H−2THP]⁺ also observed). FIG. 32 shows the structure of 6 and its mass spectrum.

N-(Morpholin-4-ylacetyl)-(3,17β-bis((tetrahydro-2H-pyran-2-yl)oxy)-7-α-[9-(4,4,5,5,5,-pentafluoropentylsulphonimidoyl)nonyl]estra-1,3,5-(10)-triene) (7): A solution of morpholine (26 μL, 300 μmol) and DIPEA (17.42 μL, 100 μmol) in DCM (1.5 mL) was added to a solution of 4 (97 mg, 100 μmol) in 1 mL DCM. The reaction was agitated for 2.5 h, diluted with 10 mL ethyl acetate, and extracted (2×water, 1×brine). The extract was dried (MgSO₄) and the solvent removed under nitrogen. The resulting amber oil was used without further purification.

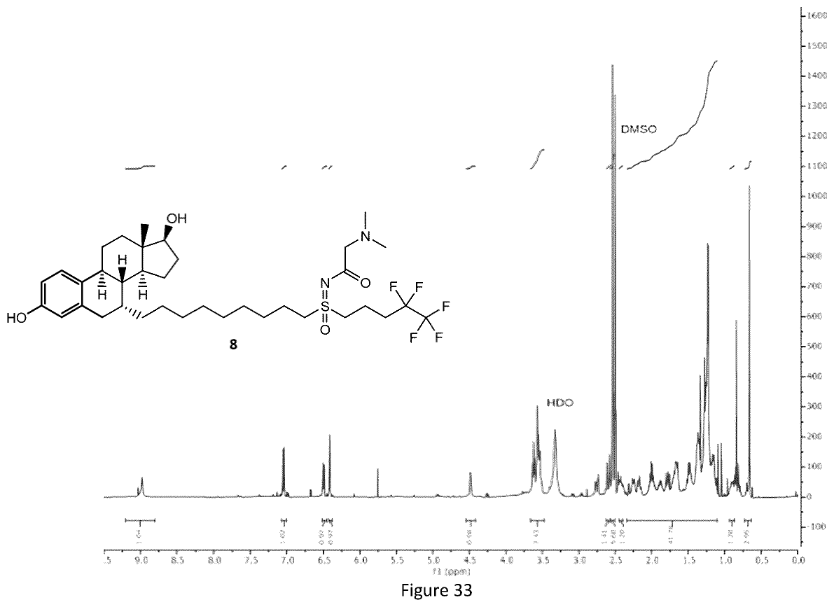

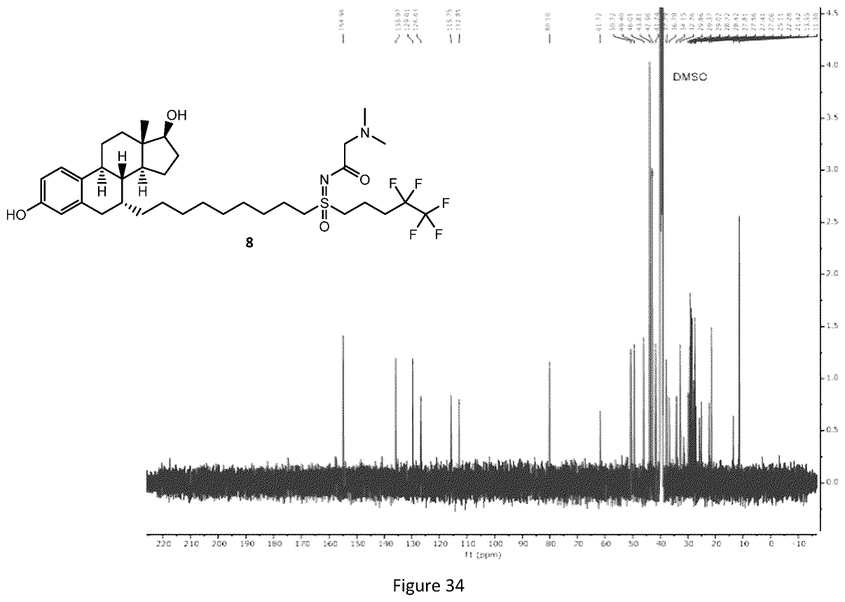

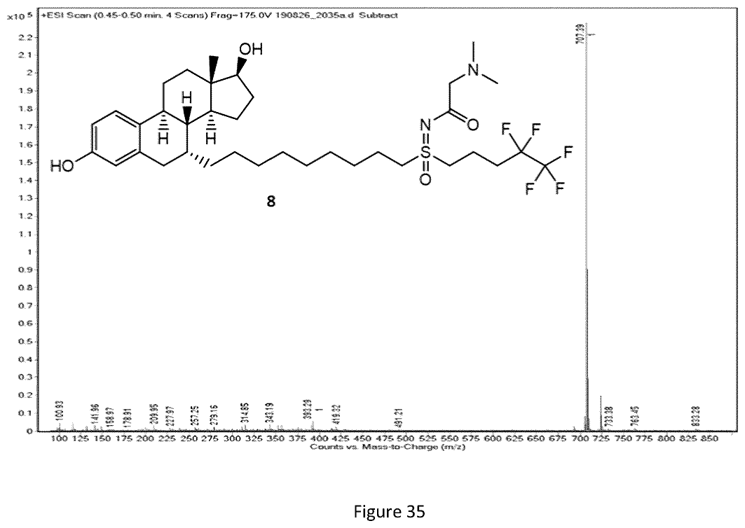

N-(2-Dimethylaminoacetyl)-(7-α-[9-(4,4,5,5,5,-pentafluoropentylsulphonimidoyl)nonyl]estra-1,3,5-(10)-triene-3,17β-diol) (8): See method for 9, but using 5 as in place of 6. ¹H NMR (500 MHz, DMSO-d6) δ 9.21-8.80 (s, 1H), 7.07-7.00 (d, 1H), 6.50 (dd, J=8.4, 2.7 Hz, 1H), 6.41 (s, J=2.6 Hz, 1H), 4.54-4.41 (m, 1H), 3.66-3.47 (m, 7H), 2.62-2.57 (m, 1H), 2.54 (s, 6H), 2.45-2.39 (m, 1H), 2.34-1.10 (m, 42H), 0.93-0.87 (m, 1H), 0.66 (s, 3H); ¹³C NMR (126 MHz, DMSO) δ 154.96, 135.97, 129.61, 126.63, 115.75, 112.85, 80.10, 61.72, 50.72, 49.40, 46.01, 43.81, 42.96, 41.74, 37.79, 36.78, 34.15, 32.76, 29.86, 29.37, 29.02, 28.72, 28.42, 27.81, 27.56, 27.41, 27.06, 25.81, 25.11, 22.28, 21.42, 13.55, 11.30; MS (ESI+) m/z [M+H]⁺ calculated for C₃₆H₅₆F₅N₂O₄S: 707.3875, found: 707.3876. FIG. 33 and FIG. 34 show, respectively, the ¹H and ¹³C NMR spectra of 8. FIG. 35 shows the mass spectrum of 8.

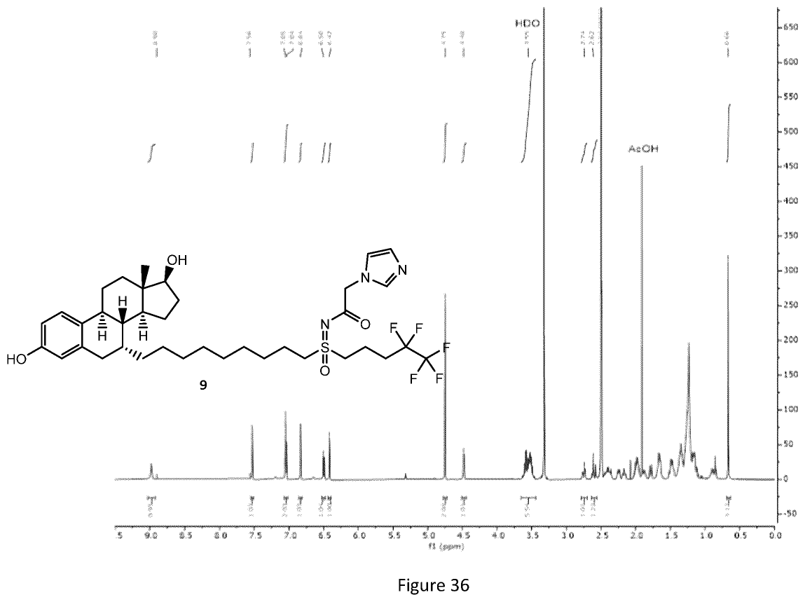

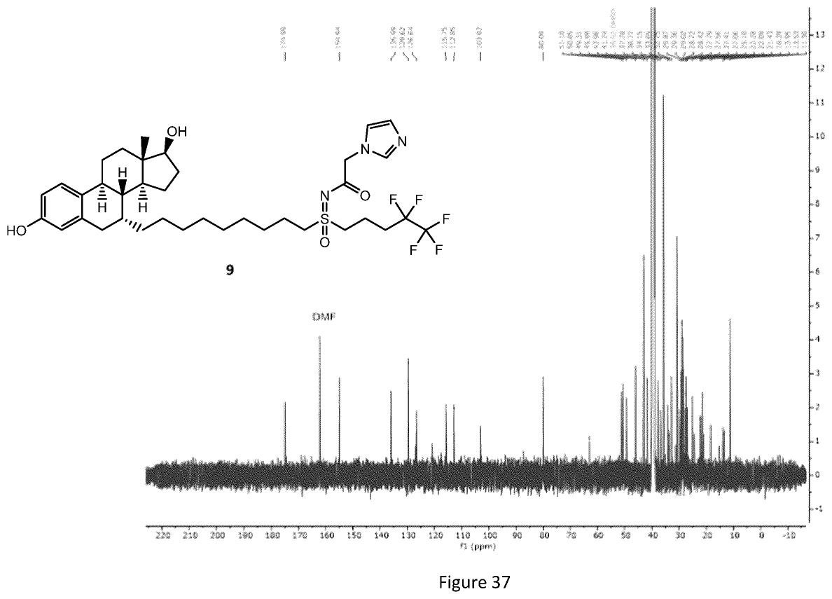

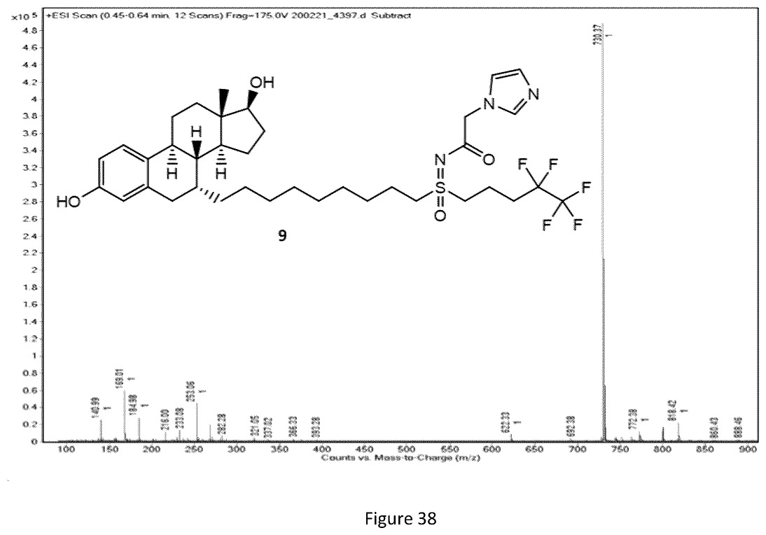

N-(1H-Imidazol-1-ylacetyl)-(7-α-[9-(4,4,5,5,5,-pentafluoropentylsulphonimidoyl)nonyl]estra-1,3,5-(10)-triene-3,17β-diol) (9): A mixture of trifluoroacetic acid (20 μL) and methanol (480 μL) was added to a solution of crude 6 (13 mg, 15 μmol) in methanol (500 μL). The mixture was agitated overnight, diluted with ethyl acetate and extracted (1× sat. Na₂CO₃, 1× brine), dried (MgSO₄), filtered, and evaporated under nitrogen. The crude product (7.9 mg) was then purified by column chromatography (silica, ethyl acetate followed by ethanol), dried under nitrogen, redissolved in acetonitrile, and filtered (0.45 μm PTFE) to yield an amber-tinted film (1.6 mg, 2.2 μmol, 15%) after drying under nitrogen. R_{f}=0.1-0.3 (acetone), 0-0.2 (ethyl acetate), 0.9 (ethanol); ¹H NMR (500 MHz, DMSO-d6) δ 8.97 (d, J=6.6 Hz, 1H), 7.64 (s, 1H), 7.09 (s, 1H), 7.04 (d, J=8.5 Hz, 1H), 6.89 (s, 1H), 6.50 (dd, J=8.4, 2.6 Hz, 1H), 6.41 (d, J=2.6 Hz, 1H), 4.77 (s, 2H), 4.48 (s, 1H), 3.65-3.43 (m, 6H), 2.46-0.78 (m, 33H), 0.66 (s, 3H); ¹³C NMR (126 MHz, DMSO-d6) δ 174.99, 154.94, 135.99, 129.62, 126.64, 115.75, 112.85, 103.02, 80.09, 62.93, 51.18, 50.65, 49.31, 45.99, 42.96, 41.74, 37.78, 36.77, 34.15, 33.65, 32.75, 29.87, 29.36, 29.02, 28.72, 28.42, 27.79, 27.56, 27.41, 27.06, 25.10, 24.48, 22.28, 22.09, 21.43, 21.05, 18.39, 13.95, 13.53, 11.30; MS (ESI+) m/z [M+H]⁺ calculated for C₃₇H₅₃F₅N₃O₄S: 730.3671, found: 730.3676. FIG. 36 and FIG. 37 show, respectively, the ¹H and ¹³C NMR spectra of 9. FIG. 38 shows the mass spectrum of 9.

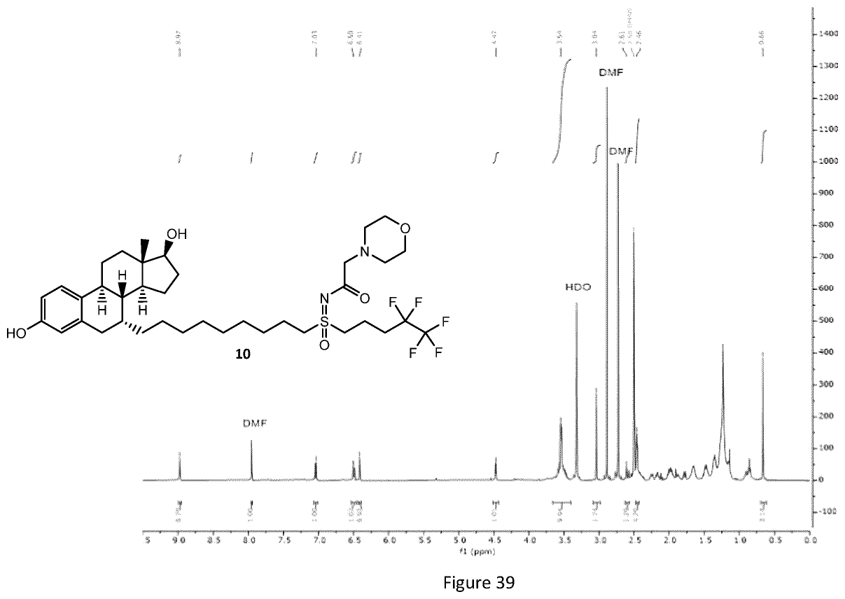

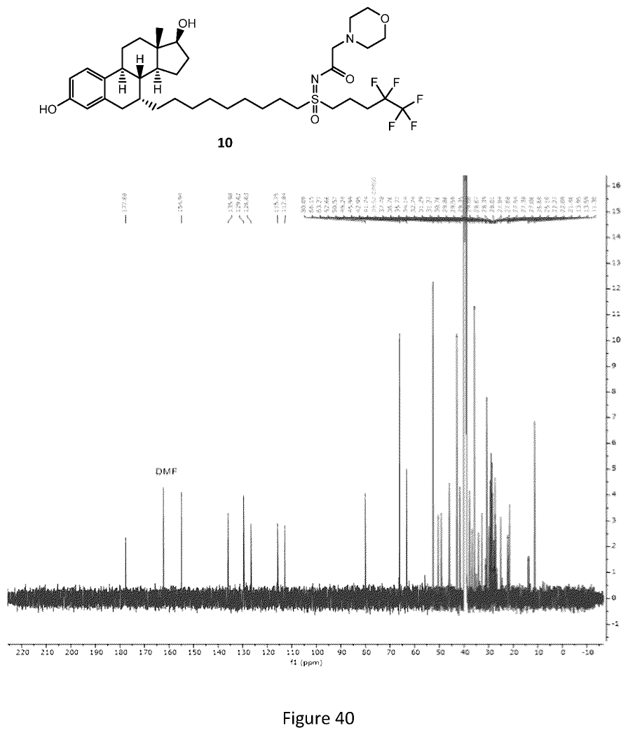

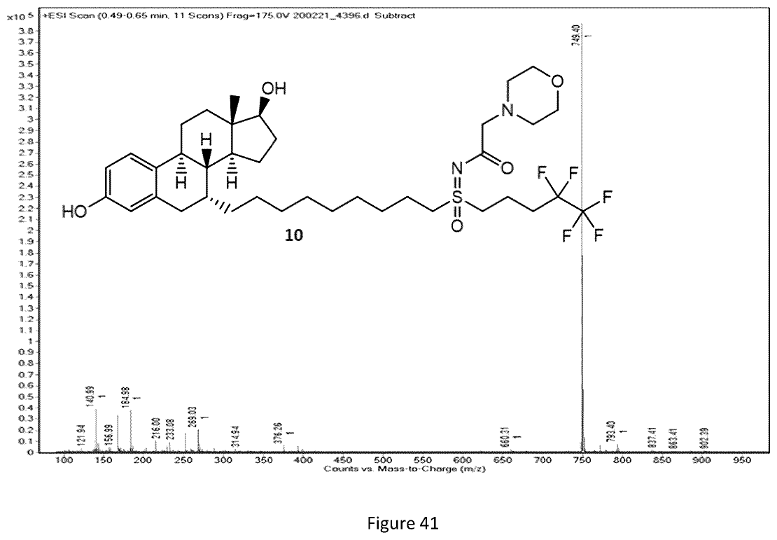

N-(Morpholin-4-ylacetyl)-(7-α-[9-(4,4,5,5,5,-pentafluoropentylsulphonimidoyl)nonyl] estra-1,3,5-(10)-triene-3,17β-diol) (10): See method for 9, but using 7 in place of 6. R_{f}=0.1 (ethyl acetate), 0.8 (acetone), 0.9 (ethanol); ¹H NMR (500 MHz, DMSO-d6) δ 8.97 (s, 1H), 7.04 (d, J=8.5 Hz, 1H), 6.50 (dd, J=8.4, 2.7 Hz, 1H), 6.41 (d, J=2.6 Hz, 1H), 4.47 (d, J=4.9 Hz, 1H), 3.66-3.40 (m, 10H), 3.04 (s, 2H), 2.60 (d, J=16.6 Hz, 1H), 2.48-2.43 (m, 4H), 2.30-0.74 (m, 32H), 0.66 (s, 3H); ¹³C NMR (126 MHz, DMSO-d6) δ 177.68, 154.94, 135.98, 129.62, 126.63, 115.75, 112.84, 80.09, 66.15, 63.27, 52.66, 50.57, 49.24, 45.99, 42.95, 41.74, 37.78, 36.76, 35.77, 34.14, 32.74, 31.29, 31.27, 30.76, 29.86, 29.59, 29.35, 29.00, 28.67, 28.39, 28.01, 27.84, 27.68, 27.54, 27.38, 27.06, 26.60, 25.10, 22.27, 22.09, 21.46, 13.95, 13.59, 11.30; MS (ESI+) m/z [M+H]⁺ calculated for C₃₈H₅₈F₅N₂O₅S: 749.3981, found: 749.3978. FIG. 39 and FIG. 40 show, respectively, the ¹H and ¹³C NMR spectra of 10. FIG. 41 shows the mass spectrum of 10.

Synthesis of Amino Acid-Modified Fulvestrant

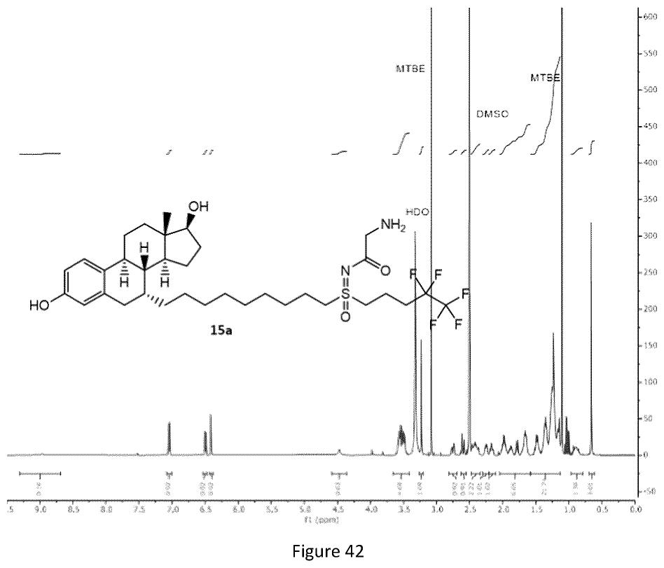

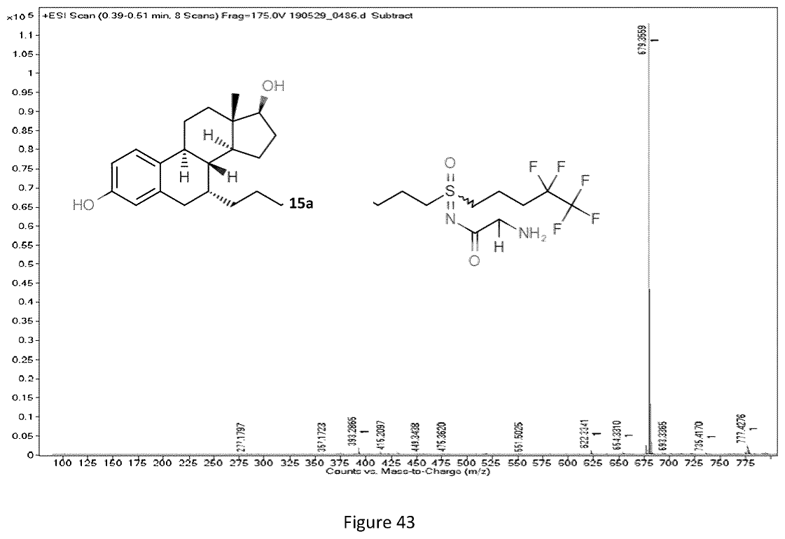

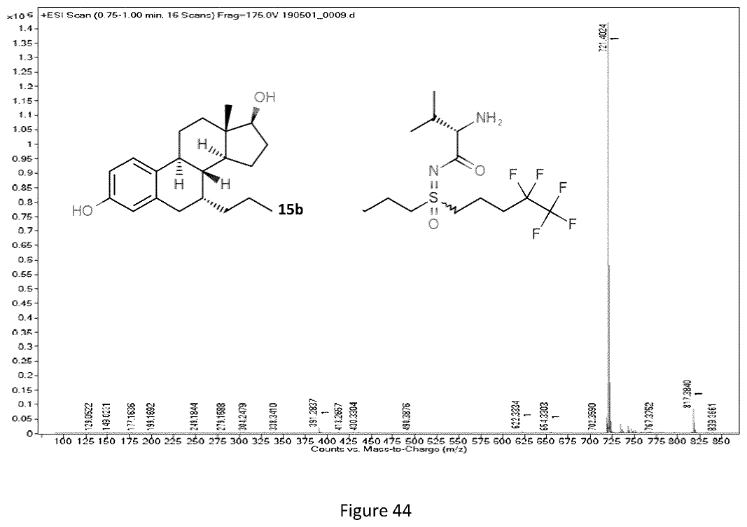

Fulvestrant was modified by addition of a glycyl or valyl group at the sulfinyl to yield 15a and 15b, respectively, as shown in FIG. 29. This modification was carried out using a 5-step procedure. First, fulvestrant (489 mg, 0.806 mmol), imidazole (538 mg, 7.90 mmol), and TBSCl (446 mg, 2.96 mmol) were dissolved in dry DMF and stirred at room temperature overnight. The solution was then diluted with MTBE and extracted with water and then saturated NaCl. The organic layer was dried over MgSO₄ and evaporated to yield species 11 in FIG. 29. Second, the TBS-protected fulvestrant was dissolved in methanol, then solid ammonium carbamate (236 mg, 3.22 mmol) and (diacetoxyiodo)benzene (712 mg, 2.42 mmol) were added under stirring. The mixture was stirred at room temperature for 4 h, then diluted with ethyl acetate and extracted with water followed by saturated NaCl. The organic layer was dried over MgSO₄ and evaporated to yield species 12 in FIG. 29. This material was purified by column chromatography using 1:1 hexanes:ethyl acetate on silica (R_{f}=0.4) to yield 547 mg of sticky oil (80% yield from 1). Third, N-Boc amino acid (60 mmol) and HCTU (25 mg, 60 mmol) were dissolved in DMF and cooled on ice. DIPEA (23 mg, 180 mmol) was added and the mixture was stirred at room temperature for 15 min. The mixture was then added to 6 (42 mg, 50 mmol) and vortexed to dissolve it, then stirred overnight. The reaction mixture was then diluted with MTBE and extracted with lightly salted water (5×) and saturated NaCl (1×). The organic layer was dried over MgSO₄ and evaporated to yield 13 as a sticky yellow oil. This material was purified by column chromatography using 3:1 hexanes:ethyl acetate on silica (R_{f}=0.4). Fourth, 13 (1 eq.) was dissolved in THF and acetic acid (5 eq.) and TBAF (5 eq) as a 1 M solution in THF were added. This mixture was heated at 60° C. for 4 h then evaporated. The resulting resin was dissolved in ethyl acetate and extracted with 0.1 M NaHCO₃ followed by saturated NaCl. The organic layer was evaporated, then purified by column chromatography using 2:1 hexanes:ethyl acetate to yield 14. Fifth, a solution containing 78% (v/v) DCM, 20% (v/v) TFA, and 2% (v/v) water was prepared and chilled on ice. This solution was added to 14 and stirred on ice for 15 min, then at room temperature for 2 h. The reaction mixture was then added dropwise to saturated, ice cold Na₂CO₃ and stirred for 15 min before diluting with water and ethyl acetate. The organic layer was isolated, dried over MgSO₄, and evaporated to yield crude 2. The product was then dissolved in acetonitrile and purified by HPLC using a 50:50 water:acetonitrile with 0.1% TFA to 100% acetonitrile with 0.1% TFA gradient on a C18-modified silica column. The resulting TFA salt of 15 was dissolved in MTBE and extracted with saturated Na₂CO₃. The organic layer was isolated, dried over MgSO₄, and evaporated to yield pure 15. FIG. 42 shows the ¹H spectrum of 15a; FIGS. 43 and 44 provide the mass spectra for 15a and 15b confirming successful synthesis of these analogues.

N-(2-aminoacetyl)-(7-α-[9-(4,4,5,5,5,-pentafluoropentylsulphonimidoyl)nonyl]estra-1,3,5-(10)-triene-3,17β-diol) (9): ¹H NMR (500 MHz, DMSO-d6) δ 7.04 (d, J=8.5 Hz, 1H), 6.50 (dd, J=8.5, 2.6 Hz, 1H), 6.41 (d, J=2.6 Hz, 1H), 4.48 (s, 1H), 3.66-3.42 (m, 5H), 3.23 (s, 2H), 2.75 (dd, J=16.6, 5.2 Hz, 1H), 2.63-2.55 (m, 1H), 2.47-2.34 (m, 2H), 2.25 (dd, J=12.1, 4.2 Hz, 1H), 2.21-2.11 (m, 1H), 2.05-1.58 (m, 7H), 1.58-1.13 (m, 22H), 0.97-0.79 (m, 1H), 0.66 (s, 3H); MS (ESI+) m/z [M+H]⁺ calculated for C₃₄H₅₂F₅N₂O₄S: 679.3562, found: 679.3559.

Colloid Formulation and Determination of CAC

Colloids were formulated as described in Example 1. Briefly, solutions of fulvestrant analogue in DMSO were prepared at various concentrations and then diluted 100-fold with PBS at different concentrations. 100 μL of each sample was analyzed by DLS and the CAC's calculated as described in Example 1.

Results

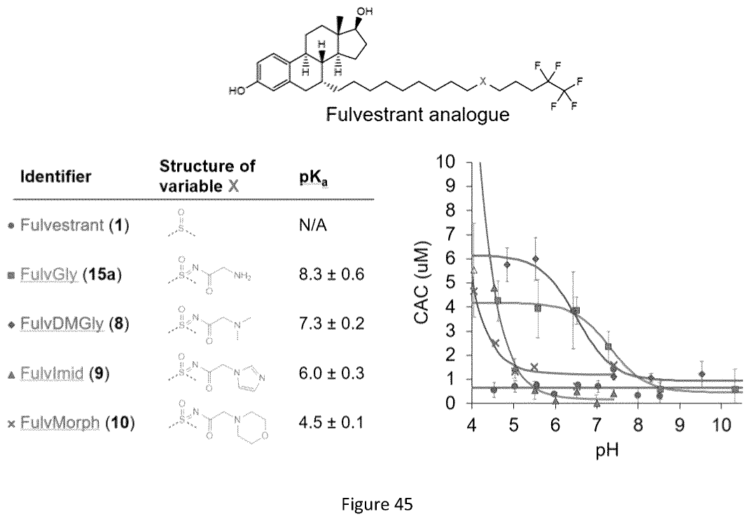

FIG. 45 shows a comparison of the CACs for fulvestrant and acid-responsive analogues of fulvestrant over a range of pH values. The increase in CAC values found for the fulvestrant analogues as the pH decreases is indicative of the acid responsiveness of the colloidal drug aggregates comprising the fulvestrant analogues. Importantly, the acid-responsive fulvestrant analogues were more soluble at lysosomal pH (4) than at extracellular pH (7.4). No change in CAC values were observed for fulvestrant with changing pH.

Discussion

As in Example 3, this study demonstrates that an aggregator that does not respond to acid can be made acid-responsive by modification with an ionizable functional group. Furthermore, these results show that the pKa of the aggregator, and thus the pH at which it becomes ionized, can be tuned by adjusting the nature of the attached ionizable functional group.

### Example 5: Siramesine Colloids

Methods

Materials

Siramesine hydrochloride was purchased from MedChemExpress. Bovine serum albumin (BSA) and dimethyl sulfoxide (DMSO) were purchased from Sigma-Aldrich. Phosphate buffered saline (PBS) was purchased from Wisent. Hydrochloric acid was purchased from VWR.

Colloid Formulation and CAC Measurement

Colloids were formulated as described in Example 1. Briefly, solutions of siramesine in DMSO were prepared at various concentrations and then diluted 100-fold with pH-adjusted PBS containing stabilizer (where applicable). 100 μL of each sample was analyzed by DLS and the CACs calculated as described in Example 1.

High Concentration Colloid Formulation

Siramesine was dissolved in DMSO at a concentration of 50 mg/mL, or in ethanol at a concentration of 10 mg/mL. 15 μL (DMSO) or 75 μL (ethanol) of this solution was diluted with 1.5 mL of water containing 5 mg/mL of BSA. The resulting colloids were flash frozen and lyophilized to yield a white cake. The lyophilized colloids were reconstituted by adding 150 μL of PBS and mixing with a pipette. The reconstituted colloids were measuring by DLS after diluting 10 μL with 90 μL of PBS.

Results

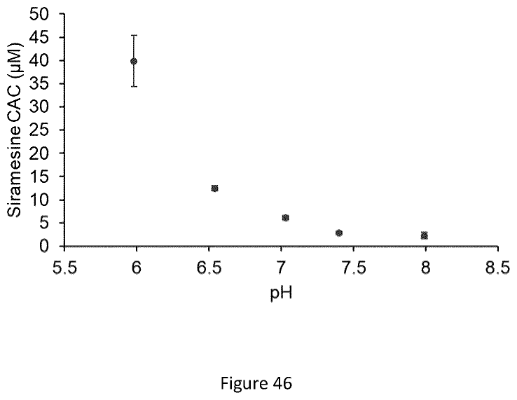

FIG. 46 shows a comparison of the CACs for siramesine over a range of pH values. The increase in CAC values found for siramesine as the pH decreases is indicative of the acid responsiveness of the colloidal drug aggregate comprising the siramesine.

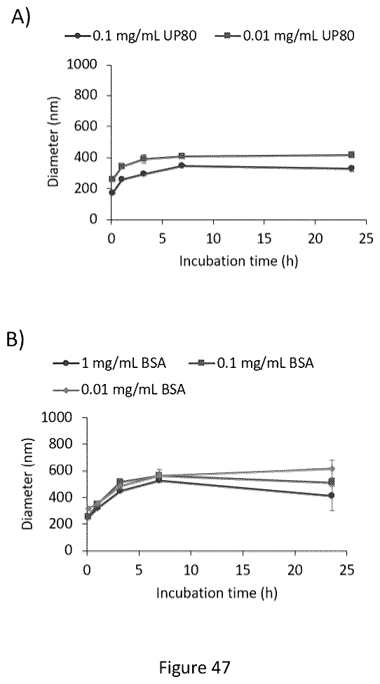

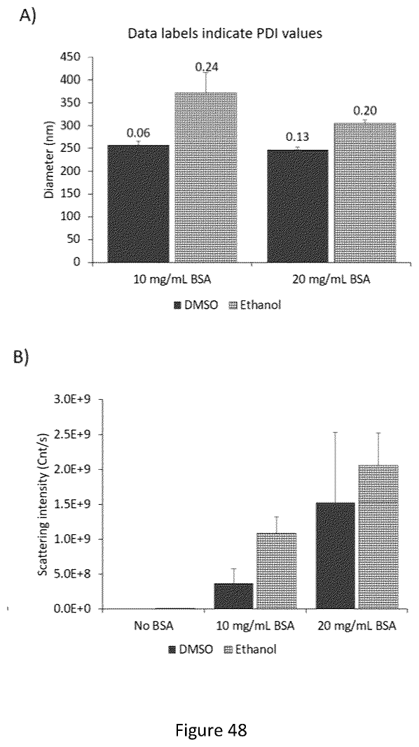

FIG. 47 and FIG. 48 demonstrate stabilization of siramesine colloids using polysorbate 80 or bovine serum albumin excipients.

Discussion

These results demonstrated that the colloid-forming compound siramesine is acid-responsive, and that colloids of siramesine can be formulated at high concentrations.

### Example 6: Diphtheria Toxin-Stabilized Sorafenib Colloids

Methods

Materials

Sorafenib was purchased from MedChemExpress. Bovine serum albumin (BSA) and RPMI 1640 cell culture media were purchased from Sigma-Aldrich. Attenuated diphtheria toxin (aDT) was obtained from Professor Roman Melnyk's (The Hospital for Sick Children, Toronto, ON, Canada). SKOV3 cells were purchased from ATCC. Fetal bovine serum was purchase from Wisent Bio Products. PrestoBlue™ cell viability reagent was purchased from Thermo Fisher Scientific.

Colloid Formulation and Characterization by Dynamic Light Scattering

Colloidal drug aggregates were formulated as described as described in Example 1 using sorafenib in place of lapatinib. In particular, colloids of sorafenib were formulated at 50 μM or 200 μM and stabilized with 10 μg/mL or 100 μg/mL diphtheria toxin in phosphate buffered saline. These formulations were assessed by dynamic light scattering as described in Example 1.

Cell Culture

SK-OV-3 cells were maintained in a humidified incubator at 37° C. with 5% atmospheric CO₂. Cells were grown in 75 cm² tissue culture flasks with 10 mL RPMI 1640 supplemented with 10% FBS, 10 UI/mL penicillin, and 10 μg mL⁻¹ streptomycin. Cells were passaged twice per week with typical subculture ratio of 1:5.

Cell Viability Experiments

After passaging, cell suspensions were diluted into fresh media and 100 μL was pipetted into each well of a 96 well plate. Five thousand cells per well were plated and allowed to adhere overnight. Then, the media was withdrawn and replaced with treatment formulations (prepared as described above) or control treatments. Cells were treated with either cell culture medium, bovine serum albumin (BSA; 100 μg/mL), aDT (100 μg/mL), BSA-stabilized sorafenib colloids (100 μg/mL BSA; 200 μM sorafenib) or aDT-stabilized sorafenib colloids (100 μg/mL aDT; 200 μM sorafenib). All treatments were prepared in complete culture media. Cells were incubated during the experiment in a humidified incubator at 37° C. with 5% atmospheric CO₂. For treatments lasting less than 3 d, the treatment solutions were removed after the prescribed time, cells were washed with fresh media, and 200 μL of fresh media was added. Cell viability was assessed 3 days after commencing treatment using the PrestoBlue™ viability assay according to the manufacturer's protocol. Cell viability was reported as a percentage of the blank (cell culture medium) control.

Results

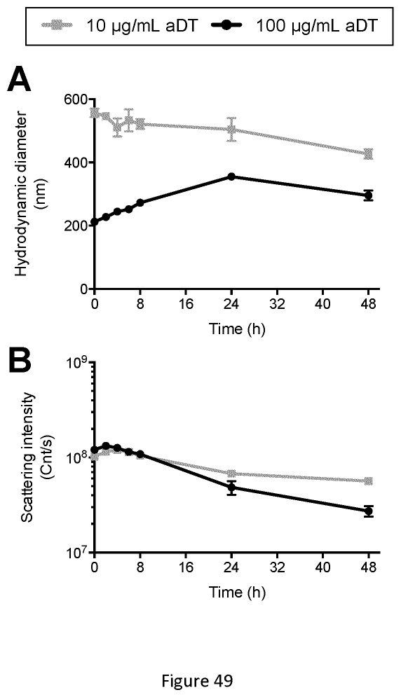

FIG. 49 shows a time course of hydrodynamic diameter of sorafenib colloids formulated with varying concentrations of aDT as a stabilizing excipient. These colloids are stable for at least 48 hours in phosphate buffered saline at 37° C. (simulating physiological conditions).

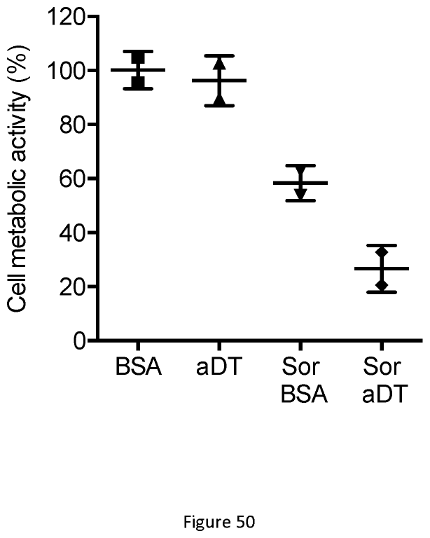

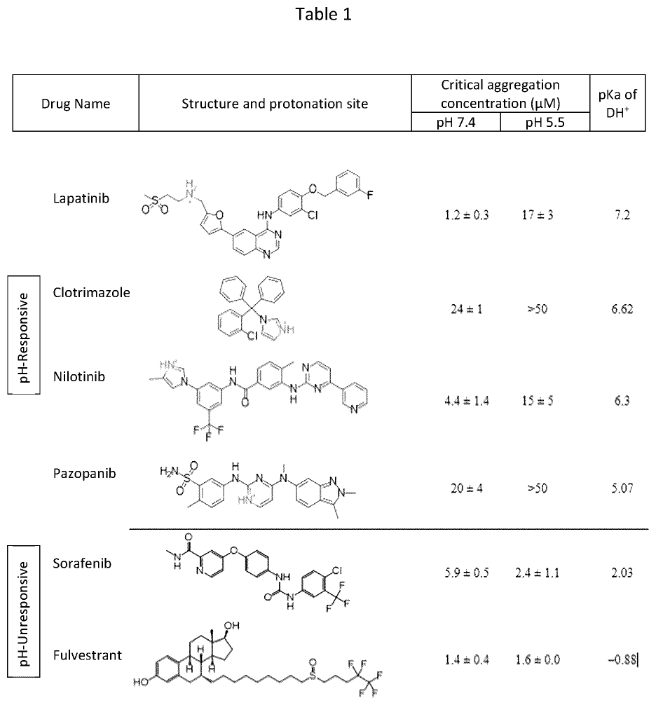

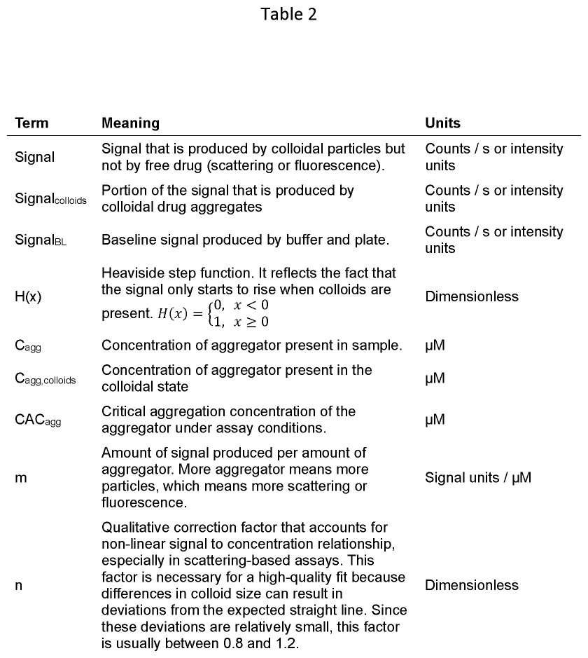

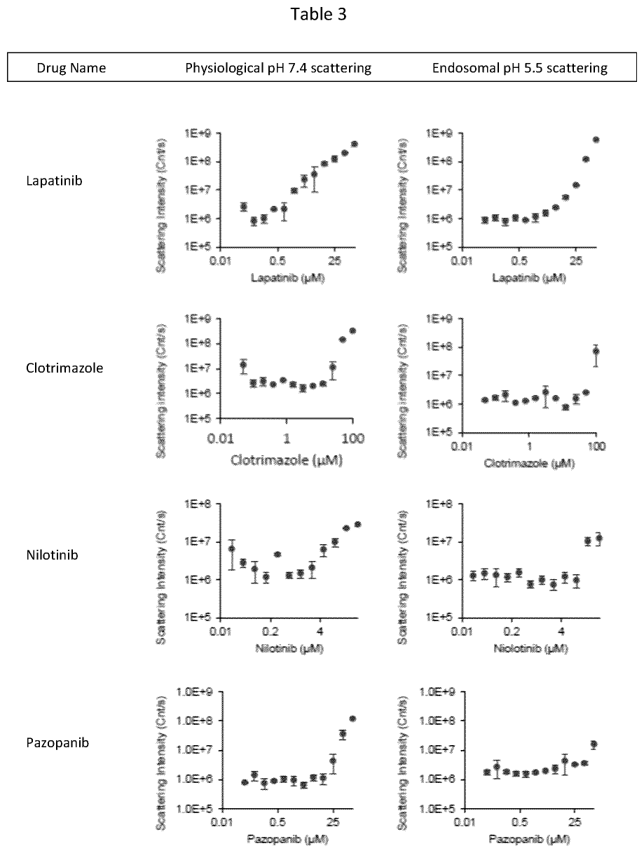

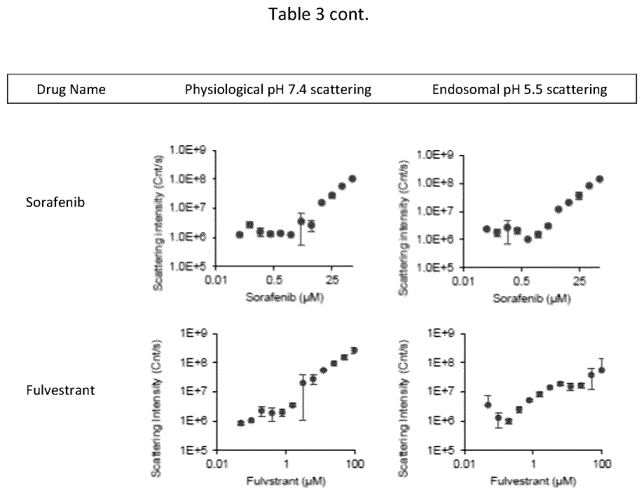

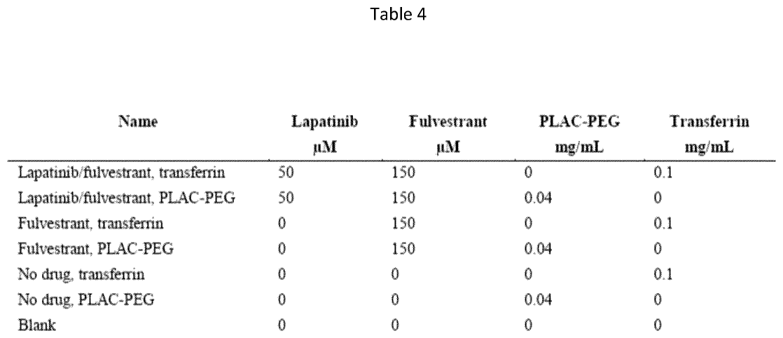

FIG. 50 demonstrates cytotoxicity of sorafenib colloids (200 μM) coated with attenuated diphtheria toxin (100 μg/mL) to an ovarian cancer cell line (SK-OV-3). aDT-stabilized sorafenib colloids (Sor-DT) reduced cell metabolic activity compared to control BSA-stabilized sorafenib colloids (Sor-BSA). Treatment with either BSA or aDT alone did not cause a reduction in cell viability.

Discussion

These results demonstrated that colloidal drug aggregates can be stabilized by an acid-responsive protein stabilizer/excipient (e.g., aDT), and this coating enhances cytotoxicity against a cancer cell line.

### REFERENCES

(1) Coan, K. E. D.; Shoichet, B. K. Stoichiometry and Physical Chemistry of Promiscuous Aggregate-Based Inhibitors. J. Am. Chem. Soc. 2008, 130 (29), 9606-9612. https://doi.org/10.1021/ja802977h.
(2) Seidler, J.; McGovern, S. L.; Doman, T. N.; Shoichet, B. K. Identification and Prediction of Promiscuous Aggregating Inhibitors among Known Drugs. J. Med. Chem. 2003, 46 (21), 4477-4486. https://doi.org/10.1021/jm030191r.
(3) Doak, A. K.; Wille, H.; Prusiner, S. B.; Shoichet, B. K. Colloid Formation by Drugs in Simulated Intestinal Fluid. J. Med. Chem. 2010, 53 (10), 4259-4265. https://doi.org/10.1021/jm100254w.
(4) Brick, M. C.; Palmer, H. J.; Whitesides, T. H. Formation of Colloidal Dispersions of Organic Materials in Aqueous Media by Solvent Shifting. Langmuir 2003, 19 (16), 6367-6380. https://doi.org/10.1021/1a034173o.
(5) Coan, K. E. D.; Maltby, D. A.; Burlingame, A. L.; Shoichet, B. K. Promiscuous Aggregate-Based Inhibitors Promote Enzyme Unfolding. J. Med. Chem. 2009, 52 (7), 2067-2075. https://doi.org/10.1021/jm801605r.
(6) McGovern, S. L.; Caselli, E.; Grigorieff, N.; Shoichet, B. K. A Common Mechanism Underlying Promiscuous Inhibitors from Virtual and High-Throughput Screening. J. Med. Chem. 2002, 45 (8), 1712-1722. https://doi.org/10.1021/jm010533y.
(7) Ganesh, A. N.; Donders, E. N.; Shoichet, B. K.; Shoichet, M. S. Colloidal Aggregation: From Screening Nuisance to Formulation Nuance. Nano Today 2018, 19, 188-200. https://doi.org/10.1016/j.nantod.2018.02.011.
(8) Ganesh, A. N.; Logie, J.; McLaughlin, C. K.; Barthel, B. L.; Koch, T. H.; Shoichet, B. K.; Shoichet, M. S. Leveraging Colloidal Aggregation for Drug-Rich Nanoparticle Formulations. Mol. Pharm. 2017, 14 (6), 1852-1860. https://doi.org/10.1021/acs.molpharmaceut.6b01015.
(9) Shamay, Y.; Shah, J.; Iik, M.; Mizrachi, A.; Leibold, J.; Tschaharganeh, D. F.; Roxbury, D.; Budhathoki-Uprety, J.; Nawaly, K.; Sugarman, J. L.; et al. Quantitative Self-Assembly Prediction Yields Targeted Nanomedicines. Nat. Mater. 2018, 17 (4), 361-368. https://doi.org/10.1038/s41563-017-0007-z.
(10) Ganesh, A. N.; Aman, A.; Logie, J.; Barthel, B. L.; Cogan, P.; Al-awar, R.; Koch, T. H.; Shoichet, B. K.; Shoichet, M. S. Colloidal Drug Aggregate Stability in High Serum Conditions and Pharmacokinetic Consequence. ACS Chem. Biol. 2019. https://doi.org/10.1021/acschembio.9b00032.
(11) Owen, S. C.; Doak, A. K.; Ganesh, A. N.; Nedyalkova, L.; McLaughlin, C. K.; Shoichet, B. K.; Shoichet, M. S. Colloidal Drug Formulations Can Explain “Bell-Shaped” Concentration—Response Curves. ACS Chem. Biol. 2014, 9 (3), 777-784. https://doi.org/10.1021/cb4007584.
(12) Owen, S. C.; Doak, A. K.; Wassam, P.; Shoichet, M. S.; Shoichet, B. K. Colloidal Aggregation Affects the Efficacy of Anticancer Drugs in Cell Culture. ACS Chem. Biol. 2012, 7 (8), 1429-1435. https://doi.org/10.1021/cb300189b.
(13) McLaughlin, C. K.; Duan, D.; Ganesh, A. N.; Torosyan, H.; Shoichet, B. K.; Shoichet, M. S. Stable Colloidal Drug Aggregates Catch and Release Active Enzymes. ACS Chem. Biol. 2016, 11 (4), 992-1000. https://doi.org/10.1021/acschembio.5b00806.
(14) Mindell, J. A. Lysosomal Acidification Mechanisms. Annu. Rev. Physiol. 2012, 74 (1), 69-86. https://doi.org/10.1146/annurev-physiol-012110-142317.
(15) Indulkar, A. S.; Box, K. J.; Taylor, R.; Ruiz, R.; Taylor, L. S. PH-Dependent Liquid-Liquid Phase Separation of Highly Supersaturated Solutions of Weakly Basic Drugs. Mol. Pharm. 2015, 12 (7), 2365-2377. https://doi.org/10.1021/acs.molpharmaceut.5b00056.
(16) Frenkel, Y. V.; Clark, A. D.; Das, K.; Wang, Y.-H.; Lewi, P. J.; Janssen, P. A. J.; Arnold, E. Concentration and PH Dependent Aggregation of Hydrophobic Drug Molecules and Relevance to Oral Bioavailability. J. Med. Chem. 2005, 48 (6), 1974-1983. https://doi.org/10.1021/jm049439i.
(17) Sugihara, H.; Taylor, L. S. Evaluation of Pazopanib Phase Behavior Following PH-Induced Supersaturation. Mol. Pharm. 2018, 15 (4), 1690-1699. https://doi.org/10.1021/acs.molpharmaceut.8b00081.
(18) Exocytosis and Endocytosis; Ivanov, A. I., Ed.; Methods in molecular biology; Humana Press: Totowa, N.J, 2008.
(19) Canton, I.; Battaglia, G. Endocytosis at the Nanoscale. Chem. Soc. Rev. 2012, 41 (7), 2718. https://doi.org/10.1039/c2cs15309b.
(20) Doherty, G. J.; McMahon, H. T. Mechanisms of Endocytosis. Annu. Rev. Biochem. 2009, 78 (1), 857-902. https://doi.org/10.1146/annurev.biochem.78.081307.110540.
(21) Wang, J.; MacEwan, S. R.; Chilkoti, A. Quantitative Mapping of the Spatial Distribution of Nanoparticles in Endo-Lysosomes by Local PH. Nano Lett. 2017, 17 (2), 1226-1232. https://doi.org/10.1021/acs.nanolett.6b05041.
(22) Ganesh, A. N.; McLaughlin, C. K.; Duan, D.; Shoichet, B. K.; Shoichet, M. S. A New Spin on Antibody—Drug Conjugates: Trastuzumab-Fulvestrant Colloidal Drug Aggregates Target HER2-Positive Cells. ACS Appl. Mater. Interfaces 2017. https://doi.org/10.1021/acsami.6b15987.
(23) Ilevbare, G. A.; Taylor, L. S. Liquid-Liquid Phase Separation in Highly Supersaturated Aqueous Solutions of Poorly Water-Soluble Drugs: Implications for Solubility Enhancing Formulations. Cryst. Growth Des. 2013, 13 (4), 1497-1509. https://doi.org/10.1021/cg301679h.
(24) Colloid Stability: The Role of Surface Forces; Tadros, T. F., Ed.; Colloids and interface science series; Wiley-VCH Verlag: Weinheim, 2007.
(25) D'Addio, S. M.; Prud'homme, R. K. Controlling Drug Nanoparticle Formation by Rapid Precipitation. Adv. Drug Deliv. Rev. 2011, 63 (6), 417-426. https://doi.org/10.1016/j.addr.2011.04.005.
(26) Bakshi, R. P.; Tatham, L. M.; Savage, A. C.; Tripathi, A. K.; Mlambo, G.; Ippolito, M. M.; Nenortas, E.; Rannard, S. P.; Owen, A.; Shapiro, T. A. Long-Acting Injectable Atovaquone Nanomedicines for Malaria Prophylaxis. Nat. Commun. 2018, 9 (1). https://doi.org/10.1038/s41467-017-02603-z.
(27) Ke, P. C.; Lin, S.; Parak, W. J.; Davis, T. P.; Caruso, F. A Decade of the Protein Corona. ACS Nano 2017. https://doi.org/10.1021/acsnano.7b08008.
(28) Indulkar, A. S.; Mo, H.; Gao, Y.; Raina, S. A.; Zhang, G. G. Z.; Taylor, L. S. Impact of Micellar Surfactant on Supersaturation and Insight into Solubilization Mechanisms in Supersaturated Solutions of Atazanavir. Pharm. Res. 2017. https://doi.org/10.1007/s11095-017-2144-0.
(29) Vermeulen, L. M. P.; Brans, T.; Samal, S. K.; Dubruel, P.; Demeester, J.; De Smedt, S. C.; Remaut, K.; Braeckmans, K. Endosomal Size and Membrane Leakiness Influence Proton Sponge-Based Rupture of Endosomal Vesicles. ACS Nano 2018, 12 (3), 2332-2345. https://doi.org/10.1021/acsnano.7b07583.
(30) Martens, T. F.; Remaut, K.; Demeester, J.; De Smedt, S. C.; Braeckmans, K. Intracellular Delivery of Nanomaterials: How to Catch Endosomal Escape in the Act. Nano Today 2014, 9 (3), 344-364. https://doi.org/10.1016/j.nantod.2014.04.011.
(31) Trasi, N. S.; Taylor, L. S. Thermodynamics of Highly Supersaturated Aqueous Solutions of Poorly Water-Soluble Drugs—Impact of a Second Drug on the Solution Phase Behavior and Implications for Combination Products. J. Pharm. Sci. 2015, 104 (8), 2583-2593. https://doi.org/10.1002/jps.24528.
(32) Knapik-Kowalczuk, J.; Tu, W.; Chmiel, K.; Rams-Baron, M.; Paluch, M. Co-Stabilization of Amorphous Pharmaceuticals—The Case of Nifedipine and Nimodipine. Mol. Pharm. 2018, 15 (6), 2455-2465. https://doi.org/10.1021/acs.molpharmaceut.8b00308.
(33) Feng, B. Y.; Shoichet, B. K. Synergy and Antagonism of Promiscuous Inhibition in Multiple-Compound Mixtures. J. Med. Chem. 2006, 49 (7), 2151-2154. https://doi.org/10.1021/jm060029z.
(34) Benjaminsen, R. V.; Mattebjerg, M. A.; Henriksen, J. R.; Moghimi, S. M.; Andresen, T. L. The Possible “Proton Sponge” Effect of Polyethylenimine (PEI) Does Not Include Change in Lysosomal PH. Mol. Ther. 2013, 21 (1), 149-157. https://doi.org/10.1038/mt.2012.185.
(35) Varkouhi, A. K.; Scholte, M.; Storm, G.; Haisma, H. J. Endosomal Escape Pathways for Delivery of Biologicals. J. Controlled Release 2011, 151 (3), 220-228. https://doi.org/10.1016/j.jconre1.2010.11.004.
(36) Amidon, G. L.; Lennernãs, H.; Shah, V. P.; Crison, J. R. A Theoretical Basis for a Biopharmaceutic Drug Classification: The Correlation of in Vitro Drug Product Dissolution and in Vivo Bioavailability. Pharm. Res. 1995, 12 (3), 413-420. https://doi.org/10.1023/A:1016212804288.
(37) Mahoney, B. P.; Raghunand, N.; Baggett, B.; Gillies, R. J. Tumor Acidity, Ion Trapping and Chemotherapeutics. Biochem. Pharmacol. 2003, 66 (7), 1207-1218. https://doi.org/10.1016/S0006-2952(03)00467-2.

(38) Smith, S. A.; Selby, L. I.; Johnston, A. P. R.; Such, G. K. The Endosomal Escape of Nanoparticles: Towards More Efficient Cellular Delivery. Bioconjug. Chem. 30.

(39) Cullis, P. R.; Hope, M. J. Lipid Nanoparticle Systems for Enabling Gene Therapies. Mol. Ther. 2017, 25 (7), 1467-1475. https://doi.org/10.1016/j.ymthe.2017.03.013.

(40) Sun, X.; Wang, G.; Zhang, H.; Hu, S.; Liu, X.; Tang, J.; Shen, Y. The Blood Clearance Kinetics and Pathway of Polymeric Micelles in Cancer Drug Delivery. ACS Nano 2018, 12 (6), 6179-6192. https://doi.org/10.1021/acsnano.8b02830.

(41) Talelli, M.; Barz, M.; Rijcken, C. J. F.; Kiessling, F.; Hennink, W. E.; Lammers, T. Core-Crosslinked Polymeric Micelles: Principles, Preparation, Biomedical Applications and Clinical Translation. Nano Today 2015, 10 (1), 93-117. https://doi.org/10.1016/j.nantod.2015.01.005.

(42) Svenson, S. What Nanomedicine in the Clinic Right Now Really Forms Nanoparticles?: What Nanomedicine Really Forms Nanoparticles. Wiley Interdiscip. Rev. Nanomed. Nanobiotechnol. 2014, 6 (2), 125-135. https://doi.org/10.1002/wnan.1257.

(43) Logie, J.; Owen, S. C.; McLaughlin, C. K.; Shoichet, M. S. PEG-Graft Density Controls Polymeric Nanoparticle Micelle Stability. Chem. Mater. 2014, 26 (9), 2847-2855. https://doi.org/10.1021/cm500448x.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. An acid-responsive composition comprising: a colloidal aggregate of one or more drugs and a stabilizing agent, wherein the colloidal aggregate disrupts, dissolves or disassembles when the acid-responsive composition is in an acid environment having a pH of less than 7.4.

2. The acid responsive composition according to claim 1, wherein the acid environment has a pH of less than about 6.5, or less than about 6, or less than about 5.5, or less than about 5, or less than about 4.5, or less than about 4.

3. The acid responsive composition according to claim 1, wherein:
(a) at least one of the one or more drugs is an ionizable drug or ionizable drug analogue, wherein the conjugate acid of the ionizable drug or drug analogue has a pKa of at least 4, or at least 4.5 or at least 5, or at least 5.5, or at least 6, or at least 6.5; and/or
(b) the stabilizing agent is an acid-responsive stabilizing agent such that the stabilizing agent undergoes a morphological and/or functional change when pH is reduced to less than about 6.5, or less than about 6, or less than about 5.5, or less than about 5, or less than about 4.5, or less than about 4.

4. The composition according to claim 1, wherein at least one of the one or more drugs is an ionizable drug or ionizable drug analogue, wherein the conjugate acid of the ionizable drug or drug analogue has a pKa of at least 4, or at least 4.5 or at least 5, or at least 5.5, or at least 6, or at least 6.5.

5. The composition according to claim 4, wherein the stabilizing agent is morphologically and/or functionally stable in acid conditions.

6. The composition according to claim 1, wherein the stabilizing agent is an acid-responsive stabilizing agent such that the stabilizing agent undergoes a morphological and/or functional change when pH is reduced to less than about 6.5, or less than about 6, or less than about 5.5, or less than about 5, or less than about 4.5, or less than about 4.

7. The composition according to claim 1, wherein the stabilizing agent is a protein (e.g. an antibody, or antibody fragment, or an attenuated diphtheria toxin), a polymer, a colloid-forming compound (e.g., vitamin E) or another colloid-forming drug (e.g., fulvestrant).

8. The composition according to claim 7, wherein the stabilizing agent is an attenuated diphtheria toxin or a derivative thereof.

9. The composition according to claim 7, wherein said other colloid-forming drug is not ionizable at a pH of 4 or less.

10. The composition according to claim 7, wherein the protein is IgG, trastuzumab, albumin, or transferrin.

11. The composition according to claim 7, wherein the polymer is a polymeric surfactant, such as UP80, PLAC-PEG, Brij 58, F127, Vitamin E-PEG, F68, or Brij L23.

12. The composition according to claim 1, wherein said one or more drugs comprises lapatinib, clotrimazole, nilotinib, pazopanib, or siramesine.

13. The composition according to claim 1, wherein when the colloidal aggregate disrupts, dissolves or disassembles when the acid-responsive composition is in the acid environment the one or more drugs are released.

14. The composition according to claim 1, wherein the acidic environment is stomach acid, or a lysosome or an endosome of a cell.

15. The composition according to claim 1, which further comprises a targeting compound for delivery of the composition to a target site.

16. The composition according to claim 15, wherein the targeting compound is a binding protein, a specific antibody or antibody fragment, or a binding molecule, wherein the targeting compound selectively binds to a cell receptor.

17. The composition according to claim 16, wherein the targeting compound is transferrin, trastuzumab, diphtheria toxin, attenuated diphtheria toxin, or a variant thereof.

18. The composition according to claim 15, wherein the targeting compound is functions together with the stabilizing agent to stabilize the colloidal drug aggregate.

19. The composition according to claim 3, wherein:
(a) the ionizable drug is lapatinib, the stabilizing agent is fulvestrant, and the composition further comprises transferrin as a targeting compound;
(b) the ionizable drug is lapatinib and the stabilizing agent is a combination of fulvestrant and a polymeric surfactant; or
(c) the drug is sorafenib and the acid-responsive stabilizer is attenuated diphtheria toxin.

20. (canceled)

21. The composition according to claim 19, the ionizable drug is lapatinib and the stabilizing agent is a combination of fulvestrant and the polymeric surfactant, wherein the polymeric surfactant is PLAC-PEG.

22. The composition according to claim 1, wherein said one or more drugs comprises an ionizable drug analogue, which comprises a drug molecule chemically modified to include an ionizable moiety.

23. The composition according to claim 22, wherein the drug molecule is sorafenib or fulvestrant.

24. The composition according to claim 22, wherein the ionizable drug analogue is pharmaceutically active or wherein the ionizable drug analogue is a prodrug and the ionizable drug moiety is cleavable following ionization.

25-27. (canceled)

28. A method for drug delivery to a target site in a subject, comprising administering to the subject an acid-responsive composition according to claim 1, wherein disruption, dissolution or disassembly of the colloidal aggregate results in delivery of at least one of the one or more drugs to the target site in the subject.
